SEQUENCE LISTING (2 sequences)

SEQ ID NO: 1 - protein, 10 aa, Artificial Sequence
  synthetic
QGGYTMHQDQ

SEQ ID NO: 2 - protein, 23 aa, Artificial Sequence
  synthetic
DRKDQGGYTMHQDQEGDTDAGLK

(19) United States
(12) Patent Application Publication
(10) Pub. No.: US 2021/0230255 A1
(43) Pub. Date: Jul. 29, 2021

(54) ANTIBODY DIRECTED AGAINST A TAU-DERIVED NEUROTOXIC PEPTIDE AND USES THEREOF

(71) Applicants: FONDAZIONE EBRI RITA LEVI-MONTALCINI, Roma (RM) (IT); CONSIGLIO NAZIONALE DELLE RICERCHE, Roma (RM) (IT)

(72) Inventors: Giuseppina AMADORO, Roma (RM) (IT); Pietro CALISSANO, Roma (RM) (IT); Veronica CORSETTI, Roma (RM) (IT)

(73) Assignees: FONDAZIONE EBRI RITA LEVI-MONTALCINI, Roma (RM) (IT); CONSIGLIO NAZIONALE DELLE RICERCHE, Roma (RM) (IT)

(21) Appl. No.: 17/050,947

(22) PCT Filed: Apr. 29, 2019
(86) PCT No.: PCT/EP2019/060934

§ 371 Date: Oct. 27, 2020

(30) Foreign Application Priority Data
Apr. 27, 2018 (IT) 102018000004936

(51) Int. Cl. C07K 16/18; A61K 39/395; A61K 45/06; A61P 25/28; A61K 39/00

(52) U.S. Cl. CPC C07K 16/18; A61K 39/3955; A61K 2039/505; A61P 25/28; A61K 45/06

(57) ABSTRACT

The present invention refers to an antibody or an antigen binding fragment thereof against a tau-derived neurotoxic peptide for use in the treatment and/or prevention of Alzheimer's disease (AD) or a non-AD tauopathy, as well as pharmaceutical compositions comprising the same.

### TECHNICAL FIELD

The present invention refers to the medical use of an antibody or an antigen binding fragment thereof against a tau-derived neurotoxic peptide, as well as pharmaceutical compositions comprising the same.

### BACKGROUND ART

Recent epidemiological studies have shown that at least 36.5 million individuals are affected worldwide by Alzheimer's disease (AD), a complex multi-factorial and progressive neurological disorder having two major pathological hallmarks: the extracellular senile plaques and intracellular neurofibrillary tangles composed of amyloid beta protein (Aβ) and hyperphosphorylated tau, respectively (Hardy and Selkoe, 2002; Reitz, 2012; Musiek and Holtzman, 2015; De Strooper and Karran, 2016). Although these insoluble and fibrillar lesions are required for the classification of full-blown AD, the initial accumulation of small, soluble diffusible forms of pathological Aβ and tau species, directly and/or indirectly, perturb the synaptic terminals (Pooler et al., 2014; Spires- and Hyman, 2014; Liao et al., 2014; Crimins et al., 2013) whose structural/functional demise is among the earliest biological correlates for the underlying memory/learning alterations occurring in AD development (Pozueta et al., 2013; Forner et al., 2017). In AD development, pathological Ab and tau, even synergically, negatively affect the neuronal network activity at vulnerable synapses into limbic area (Braak and Braak, 1996; Morrison and Hof, 2002; Fu and Duff, 2018; Jagust W, 2018), leading to memory/learning dysfunction prior to overt and wide neuronal loss (Kametani and Hasegawa et al., 2018; Crimins et al., 2013; Spires-Jones et al., 2014). In this context, treatment of the hippocampal-entorhinal circuitry at early-middle stages of pathology progression—when extensive neurodegeneration has not yet developed—turns out to be the most effective in preventing the disease-associated brain atrophy and related cognitive impairment (Bokde et al., 2009). However, since the age represents the main risk factor for AD, its prevalence is expected to increase exponentially with population aging and no effective therapeutic agent is nowadays available to slow down and/or delay the disease progression (Mangialasche et al., 2010; Alzheimer's Association. 2018). Compelling clinical and experimental studies have demonstrated that: (i) tau is a pivotal driver of neurodegeneration since pure amyloidosis is asymptomatic (Murray et al., 2015) and the Aβ-driven neurotoxicity is tau-dependent both in cellular and animals AD models (Rapoport et al., 2002; Roberson et al., 2007; King et al., 2006; Vossel et al., 2010; Shipton et al., 2011; Ittner et al., 2010; Nussbaum et al., 2012; Bloom, 2014); (ii) tau-laden neurofibrillary tangles, but not the AP-based senile plaques, better correlate with the degree of synaptic failure (Falke et al., 2003; Ingelsson et al., 2004; Serrano-Pozo et al., 2011) and with the clinical progression of the disease symptoms (Brier et al., 2016; Nelson et al., 2012; Murray et al., 2015; Xia C. et al., 2017; Zhou J. et al., 2012); (iii) independently of its ability of seeding aggregation, pathological extracellular tau is per se neurotoxic (Diaz-Hernandez et al., 2010; Medina and Avila et al. 2014a-b; Hu et al., 2018) and propagates trans-synaptically along interconnected neuronal network brains in a stereotypical manner which strongly correlates with the development of clinical symptoms in AD progression (Pooler et al., 2013; Mohamed et al., 2013; Yamada et al., 2017). These pathologically-relevant findings represent the rationale which strongly advocates the employment of tau-based vaccination (Li and Gotz, 2017; Congdon and Sigurdsson, 2018) as promising disease-modifying therapeutic advance for the treatment of human neurodegenerative tauopathies, including AD (Novak et al., 2018). After initial studies showing the feasibility of a passive immunization approach to tau pathology in mouse models (Chai et al., 2011; Yanamandra et al., 2013; d'Abramo et al., 2013; Castillo-Carranza et al., 2014; Boutajangout et al., 2011), current tau-based immunotherapy programs are under way in clinical trials on human beings (Congdon and Sigurdsson, 2018; Pedersen and Sigurdsson, 2015: Li and Gotz, 2017) representing an actual alternative to the not-encouraging Aβ-based pharmacological and immunological approaches (Agadjanyan et al., 2017; Sigurdsson, 2008; Schroeder et al., 2016; Doody et al. 2014; Salloway et al. 2014; Giacobini and Gold, 2013). In the last years, truncation at N-terminal domain of tau has become attractive for the preclinical development of curative anti-tau antibodies given its important early role in both neurofibrillary aggregation and neurodegeneration occurring in human tauopathies, including AD (Guillozet-Bongaarts et al., 2007; Garcia-Sierra et al., 2008). On one hand, tau cleavage may generate amyloidogenic fragments that initiate its aggregation which, in turn, can cause toxicity (Wang and Mandelkow, 2010). On the other hand, tau proteolysis may result in production of noxious truncated species which drive neurodegeneration as a result of their deleterious action on pre- and/or post-synaptic functions and/or their secretion transcellular propagation, independently of aggregative pathway(s) and in a fragment-dependent manner (Quinn et al., 2018). To this regard, recent in vitro and in vivo data have highlighted a crucial role of proteolytic tau fragments, in intracellular or extracellular form(s), in the initiation/progression of AD paving thus the way for their potential use as biomarkers for diagnosing dementia and/or monitoring disease progression and as therapeutic targets (Avila et al., 2016; Sebastián-Serrano et al., 2018). Extracellular cleaved tau is toxic to neurons by increasing the Ab production (Bright et al., 2015) and/or by impairing synaptic plasticity (Florenzano et al., 2017; Borreca et al., 2018; F A et al., 2016; Hu N W et al. 2018). Hyperphosphorylation and caspase-3 cleavage of tau (Asp421), which promote aggregation, also favor the protein secretion in vitro (Plouffe et al., 2012). The amino-terminal projection domain of human tau—which interacts with the plasma membrane (Brandt et al., 1995) and undergoes early conformational changes in AD and other human tauopathies (Combs et al., 2016, 2017)—is endowed with deleterious action, mainly at nerve endings (Ittner et al., 2010; King et al., 2006 Amadoro et al., 2012; Zho et al., 2017). The N-terminus extremity of tau lacking the microtubule binding domains is prone to come into higher order oligomerization (Feinstein et al., 2016) and is required and specifically secreted to the extracellular space in in situ tauopathy model (Kim et al., 2010) and in induced pluripotent stem cell (iPSC)-derived human neurons (Sato et al., 2018). Soluble and unaggregated C-terminally truncated tau species are also preferentially secreted from synaptosomes of AD brains (Sokolow et al., 2015) and in conditioned media from patient-derived induced pluripotent stem cells (iPSC) cortical neurons of affected subjects (Bright et al., 2015; Kanmert et al., 2015; Sato et al., 2018).

Interestingly, although full-length tau is found in cerebral spinal fluid (CSF) from healthy humans, CSF-tau is mainly detected in AD patients as a heterogeneous population of fragments, including the NH₂-terminal and/or prolin-rich domain of protein (Meredith et al., 2013; Johnson et al., 1997; Portelius et al., 2008; Amadoro et al., 2014; Cicognola et al., 2018; Chen Z et al., 2018). Exosomes-associated NH₂ derived tau fragments are also detected in CSF from AD patients (Saman et al., 2012) and a different CSF pattern of NH₂-derived tau fragments may reflect disease-specific neurodegenerative processes (Borroni et al., 2009). Consistently, passive immunotherapy with antibody targeting the N-terminal projection domain of full-length human tau has shown to be beneficial in improving the cognitive deficits (Yanamandra et al., 2013; Dai et al., 2015; Subramanian et al., 2017) and in preventing the seeding/spreading of tau pathology (Dai et al., 2018) in AD transgenic mice. Both intracerebroventricular (i.c.v.) infusion and peripheral administration of anti-tau antibodies specific for N-terminal 25-30 epitopes are curative in P301S mice model of tauopathy, by preventing the brain atrophy and improving the motor/sensorimotor functions (Yanamandra et al., 2013, 2015). Immunization with antibody directed against the N-terminal end of full-length tau protein (Dai et al., 2017) significantly reduced the level of amyloid precursor protein (APP), Aβ40 and Aβ42 in CA1 region of AD animal models, indicating that tau-based immunotherapy is actually able to restore the Aβ-dependent and/or independent synaptic dysfunction(s) occurring in early AD and in other related tauopathies associated to dementias (Panza et al., 2016; Pedersen and Sigurdsson, 2015). However, albeit the main factor underlying the development and progression of AD is tau, being AB removal per se insufficient for an effective disease modification (Kametani and Hasegawa et al., 2018), the tau expression at physiological level is required for normal neuronal functions underlying the learning/memory plasticity (Pooler et al., 2014; Regan et al., 2017) and its downregulation, even if moderate, has been proved to have deleterious effects, both in vitro and in vivo (Biundo et al., 2018; Velazquez and Oddo, 2018). In this framework, the identification of the molecular nature of extracellular and soluble/diffusible noxious tau species which are causally involved in synaptic failure at pre-symptomatic stages of AD turns out to be of central importance for developing an effective better-targeted and, then, a more effective tau-based immunotherapeutic approach aimed to mitigate, or delay, the cognitive deficits associated with AD and other human tauopathies. Finally, the development and in vivo characterization of tau-directed antibodies which selectively engage the soluble neurotoxic species, without cross-reaction towards the physiological full-length form may have a unique therapeutic advantage leading to beneficial therapeutical effects in the absence of unwanted consequences due to “loss of function” of tau protein (Kontsekova et al., 2014). In search of specific epitopes located on N-terminus of pathogenic tau which could be antibody-targetable for therapeutic treatment of human tauopathies, inventors have previously reported that the pathologically-relevant NH₂tau 26-44—which is the minimal active moiety of neurotoxic 20-22 kDa NH₂-derived tau peptide (aka NH2htau) accumulating in vivo at AD presynaptic terminals (Amadoro et al., 2006, 2010, 2012, Corsetti et al., 2015) and present in CSFs from living patients suffering from AD and other not-AD neurodegenerative diseases (Amadoro et al., 2014) is able to negatively impact on normal synaptic function(s) in vitro (Florenzano et al., 2017) and in vivo (Borreca et al., 2018). As further confirmed by other research groups (Cicognola et al., 2018; Sokolow et al. 2015, Barthélemy et al., 2016; Bright et al., 2015; Sato et al., 2018), the tau-based vaccination selectively targeting the AD-linked NH₂-derived tau species may have important clinical and translational implications in contrasting the early neuropathological and cognitive alterations of subjects affected from human AD and non-AD tauopathies. By intravenous (i.v.) administration in both Tg2576 and 3XTg AD transgenic animals mice, here inventors explored the potentially-beneficial immunotherapeutic power of the 12A12 mAb, a cleavage-specific neoepitope antibody (Amadoro et al., 2012) recognizing the human tau truncation at D25 (DRKD₂₆QGGYTMHQDQEGDTDAGLK₄₄ (SEQ ID NO:2)), a known N-terminal truncation protein site (Quinn et al., 2018) previously identified in cellular and animal AD models (Corsetti et al., 2008) and in human AD brains (Rohn et al., 2002). Importantly, unlike other both murine and humanized NH₂tau-directed immunotherapeutical antibodies (Dai et al., 2015, 2017, 2018; Yanamandra et al., 2013, 2015; Subramanian et al., 2017; Qureshi et al., 2018), 12A12mAb is able to react only against the 20-22 kDa neurotoxic NH₂-truncated tau (aka NH₂htau) but not the physiological full-length form of protein (Amadoro et al., 2012; Corsetti et al., 2008). However, there is still the need for therapeutic tools that target toxic NH₂-derived tau fragments, in particular for the treatment of AD and other non-AD tauopathies.

### SUMMARY OF THE INVENTION

In the present invention, Tg2576 and 3XTg transgenic mice were used. They represent two established AD animals models which express the human APP695 with Swedish mutations (K670N-M671L) (Hsiao et al., 1996) or the same mutation in combination with MAPT P301L and PSEN1 M146V (Oddo et al., 2003), respectively. Such models display a marked accumulation of the NH₂htau fragment into pathological-relevant vulnerable limbic regions which are known to be affected by neurofibrillary tau changes at early stages of disease (Braak and Braak 1991). Tg2576 and 3xTg mice are cognitively normal at 1-3 months of age and cognitive performance declines from the age of 5-6 months onward (Dineley et al. 2002; Westerman et al. 2002; Oddo et al., 2003). The cleavage-specific 12A12mAb selectively binds the neurotoxic AD-linked NH₂ 26-230 human tau fragment and does not cross-react with the full-length physiological form of tau. The inventors show that intravenous (i.v.) administration of a cleavage-specific 12A12 monoclonal antibody (mAb) which targets the proximal 26-36 aa stretch encompassing the extreme N-terminal domain of human tau (14 days treatment; 60 μg 12A12 mAb/mouse/week) in aging (symptomatic) Tg2576 and 3XTg transgenic mice showing progressive accumulation of the neurotoxic NH₂htau into hippocampus is able:

(i) to downregulate the early pathological alterations of both tau and amyloid metabolisms which are causally involved in synaptic and cognitive impairment associated with the development of AD phenotype (Kametani et al., 2018; Spires-Jones et al., 2014). Interestingly the antibody does not change the steady-state level of physiological full-length Tau protein;

(ii) to offer a significant neuroprotection in two different hippocampal-based behavioural tasks (Novel object recognition (NOR) and Object Place Recognition (OPR)) aimed at evaluating the first type of learning/memory to be early and severely impaired in affected subjects (Grayson et al. 2015; Reed et al., 1997; Zola et al., 2001).

(iii) to restore in immunized AD animals the local specific upregulation of activity-regulated cytoskeleton-associated protein Arc which is normally evoked by short-term memory/learning task.

(iv) to prevent in vaccinated Tg-AD mice, when compared with wild-type age-matched littermates, the AD-related hippocampal impairments in electrophysiological recordings of LTP (long term potentiation) known to be the biological correlate of mnesic plasticity.

(v) to rescue in immunized Tg-AD animals from both genetic backgrounds the loss in hippocampal dendritic spine density which are the sites of excitatory synapses and, then, the cellular morphological specializations devoted to memory-forming processes in neurons.

(vi) to downregulate the levels of two inflammatory astroglial and microglial markers such as the glial fibrillary acidic protein (GFAP) and Iba1, indicative of reactive gliosis.

Taken together, these biological, behavioural, morphological and electrophysiological findings strongly demonstrate for the first time the immunotherapeutic power of the 12A12 mAb in vivo in two different AD transgenic animal models fostering, thus the use of its humanized version in contrasting the early neuropathological and cognitive alterations of subjects affected from human AD and non-AD tauopathies;

Finally, interventions that improve the cognition by concomitantly reducing both Aβ and tau pathology appears to be endowed with higher potentialities in successfully treating the AD symptomatology than those altering either pathology alone (Lambracht-Washington and Rosenberg, 2013). In this framework, passive immunization with our cleavage specific 12A12mAb—which reduces both Abeta and tau pathology without cross-reaction with the physiological full-length tau protein and without potential adverse side-effects—can be a promising disease-modifying approach to cure human tauopathies.

The present antibody reverses phenotypic pathological features present in two transgenic models of AD such as tau hyperphosphorylation, amyloidosis and cognitive impairments.

Further the antibody of the present invention restores in immunized AD animals the specific upregulation of activity-regulated cytoskeleton-associated protein Arc which is normally evoked by short-term memory/learning task.

The cleavage-specific antibody of the present invention selectively binds the neurotoxic AD-linked NH₂ 26-230 human tau fragment, as assessed by Western blotting analysis and ELISA test. The antibody of the present invention does not cross-react in vivo with the full-length physiological form of tau, as assessed after its inoculation in both AD transgenic animals, leading to beneficial therapeutical effects in the absence of unwanted consequences due to “loss of function” of normal tau.

Therefore, the present invention provides a monoclonal antibody, or an antigen binding fragment thereof, that binds to an antigen comprising the sequence QGGYTMHQDQ (SEQ ID No. 1) and possesses at least one biological activity selected from: inhibition of pathological hyperphosphorylation of Tau, reduction of the most neurotoxic amyloid precursor protein (APP)-derived amyloid-beta species (monomer and low-molecular weight oligomers), increase in task-induced Arc expression when compared to a proper control, significant neuroprotection in at least one of two different hippocampal-based behavioural tasks (Novel object recognition (NOR) and Object Place Recognition (OPR)), prevention of the loss in dendritic spine density, reduction of neuroinflammation, normalization of LTP changes, for use in the treatment and/or prevention of Alzheimer's disease (AD) or a non-AD tauopathy. It is relevant to note that the present monoclonal antibody is able to inhibit in vivo not only the tau—but also the amyloid-dependent pathology by attenuating the site-specific hyperphosphorylation of tau, the production of the most neurotoxic amyloid precursor protein (APP)-derived Aβ species (monomer and low-molecular weight oligomers), as assessed by Western blotting analysis with specific commercial antibodies (AT8,6E10) on hippocampal extracts from immunized AD transgenic mice of two different genetic backgrounds (Tg2576, Tg3X) in comparison to wild-type saline-treated controls. The in vivo immunotherapeutic action of this present monoclonal antibody in successfully improving the cognitive impairment of AD transgenic mice is also confirmed by the positive modulation in Arc expression—an activity-regulated cytoskeletal (Arc) gene which is critical for consolidating memory—whose synaptic level is increased from immunized and trained group in comparison to wild-type saline-treated controls, as assessed by Western blotting analysis on synaptosomal fractions with specific commercial antibody (C-7).

Preferably the monoclonal antibody, or an antigen binding fragment thereof does not change full-length tau levels when compared to a proper control.

Preferably the monoclonal antibody, or an antigen binding fragment thereof binds to an antigen consisting of the sequence QGGYTMHQDQ (SEQ ID No. 1).

Preferably said antibody or antigen binding fragment thereof comprises at least one human constant region. Preferably said constant region is the human IgGI/lgKappa constant region.

Preferably said antibody or antigen binding fragment thereof is a humanized or resurfaced antibody.

Still preferably said antibody or antigen binding fragment thereof is a Fab, Fab′, F(ab′)2 or Fv fragment.

More preferably said antibody is a bispecific antibody.

The present invention provides a conjugate comprising the antibody or antigen binding fragment as defined above.

Preferably AD is a genetic or sporadic form.

The invention further provides a pharmaceutical composition comprising the monoclonal antibody, or an antigen binding fragment thereof or the conjugate of the invention and proper excipients for use in the treatment of Alzheimer's disease (AD) or a non-AD tauopathy.

Preferably the pharmaceutical composition further comprises a therapeutic agent.

Preferably the therapeutic agent is selected from the group consisting of: Tau Aggregation/oligomerization Inhibitors (TRxO237); Kinase Inhibitors and Phosphatase Activators (saracatinib—AZD0530; Tideglusib—NP31112, NP-12); Microtubule Stabilizers (TPI-287; Davunetide (NAP; AL-108); activators of autophagy and proteasome-mediated clearance (rapamycin; trehalose); reactive oxygen species (ROS) inhibitors (omega-3 fatty acid docosahexaenoic acid (DHA) curcumin; vitamin E, vitamin C, lipoic acid and coenzyme Q); mitochondrial function enhancers; active (AADvac-1; ACI-35) and passive vaccination (RG6100 (also known as R07105705); ABBV-8E12 (also known as C2N-8E12) (Li et al., 2017; Medina, 2018 all incorporated by reference).

The term “antibody” is used herein in the broadest sense and specifically covers monoclonal antibodies (including full length monoclonal antibodies) of any isotype such as IgG, IgM, IgA, IgD and IgE, multispecific antibodies, chimeric antibodies, and antibody fragments. An antibody reactive with a specific antigen can be generated by recombinant methods such as selection of libraries of recombinant antibodies in phage or similar vectors, or by immunizing an animal with the antigen or an antigen-encoding nucleic acid.

A typical IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus. The more highly conserved portions of the variable regions are called the “framework regions”.

As used herein, “VH” or “VH” refers to the variable region of an immunoglobulin heavy chain of an antibody, including the heavy chain of an Fv, scFv, dsFv, Fab, Fab′ or F(ab′)2 fragment. Reference to “VL” or “VL” refers to the variable region of the immunoglobulin light chain of an antibody, including the light chain of an Fv, scFv, dsFv, Fab, Fab′ or F(ab′)2 fragment. animal.

A “monoclonal antibody”, as used herein, is an antibody obtained from a population of substantially homogeneous antibodies, i.e. the antibodies forming this population are essentially identical except for possible naturally occurring mutations which might be present in minor amounts. These antibodies are directed against a single epitope and are therefore highly specific.

An “epitope” is the site on the antigen to which an antibody binds. If the antigen is a polymer, such as a protein or polysaccharide, the epitope can be formed by contiguous residues or by non-contiguous residues brought into close proximity by the folding of an antigenic polymer. In proteins, epitopes formed by contiguous amino acids are typically retained on exposure to denaturing solvents, whereas epitopes formed by noncontiguous amino acids are typically lost under said exposure. As used herein, the term “K” refers to the dissociation constant of a particular antibody/antigen interaction.

The scope of the present invention is not limited to 12A12 antibody and fragments thereof. Instead, all antibodies and fragments that specifically bind to an antigen comprising the sequence QGGYTMHQDQ (SEQ ID No. 1) and that possesses at least one biological activity selected from inhibition of pathological hyperphosphorylation of Tau, reduction of the most neurotoxic amyloid precursor protein (APP)-derived amyloid-beta species (monomer and low-molecular weight oligomers), increase in task-induced Arc expression when compared to a proper control, significant neuroprotection in at least one of two different hippocampal-based behavioural tasks (Novel object recognition (NOR) and Object Place Recognition (OPR)), prevention of the loss in dendritic spine density, reduction of neuroinflammation, normalization of LTP changes fall within the scope of the present invention. Thus, antibodies and antibody fragments may differ from antibody 12A12 or the humanized derivatives in the amino acid sequences of their scaffold, CDRs, light chain and heavy chain, and still fall within the scope of the present invention.

The antibody according to the invention also include antibodies that specifically bind to an antigen comprising (or consisting of) a sequence having a % of identity of at least 70%, 75%, 80%, 85%, 86%, 85%, 88%, 89%, 90%, 91%, 92%, 93%, 94%, 95%, 96%, 97%, 98% or 99% or 100% with the sequence QGGYTMHQDQ (SEQ ID No. 1).

As used herein, a “chimeric antibody” is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass. Methods for producing chimeric antibodies are known in the art. See, e.g., Morrison, 1985, Science, 229: 1202; Oi et al., 1986, BioTechniques, 4: 214; Gillies et al., 1989, J. Immunol. Methods, 125: 191-202; U.S. Pat. Nos. 5,807,715; 4,816,567; and 4,816,397, which are incorporated herein by reference in their entireties.

In one embodiment of the invention, chimeric versions of 12A12 are provided. In particular, said chimeric versions contain at least one human constant region. In a more preferred embodiment, this human constant region is the human lgG1/Kappa constant region.

The term “humanized antibody”, as used herein, refers to a chimeric antibody which contain minimal sequence derived from non-human immunoglobulin. The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. As used herein, the resurfacing technology uses a combination of molecular modeling, statistical analysis and mutagenesis to alter the non-CDR surfaces of antibody variable regions to resemble the surfaces of known antibodies of the target host. The CDR grafting technology involves substituting the complementarity determining regions of, for example, a mouse antibody, into a human framework domain, e.g., see WO 92/22653. Humanized chimeric antibodies preferably have constant regions and variable regions other than the complementarity determining regions (CDRs) derived substantially or exclusively from the corresponding human antibody regions and CDRs derived substantially or exclusively from a mammal other than a human.

Strategies and methods for the resurfacing of antibodies, and other methods for reducing immunogenicity of antibodies within a different host, are disclosed in U.S. Pat. No. 5,639,641, which is hereby incorporated in its entirety by reference. Briefly, in a preferred method, (1) position alignments of a pool of antibody heavy and light chain variable regions is generated to give a set of heavy and light chain variable region framework surface exposed positions wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 A of any atom of any residue of the complementarity-determining regions of the rodent antibody; and (5) the humanized rodent antibody having binding specificity is produced. Antibodies can be humanized using a variety of other techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., 1991, Molecular Immunology 28(4/5): 489-498; Studnicka G. M. et al., 1994, Protein Engineering, 7(6): 805-814; Roguska M A et al., 1994, PNAS, 91: 969-973), and chain shuffling (U.S. Pat. No. 5,565,332). Human antibodies can be made by a variety of methods known in the art including phage display methods. See also U.S. Pat. Nos. 4,444,887, 4,716,111, 5,545,806, and 5,814,318; and international patent application publication numbers WO 98/46645, WO 98/50433, WO 98/24893, WO 98/16654, WO 96/34096, WO 96/33735, and WO 91/10741 (said references incorporated by reference in their entireties).

The present invention provides humanized antibodies or fragments thereof, which specifically bind to an antigen comprising the sequence QGGYTMHQDQ (SEQ ID No. 1) and that possesses at least one biological activity selected from inhibition of pathological hyperphosphorylation of Tau, reduction of the amyloid precursor protein (APP)-derived neurotoxic amyloid-beta species (monomer and low-molecular weight oligomers), increase in task-induced Arc expression when compared to a proper control, significant neuroprotection in at least one of two different hippocampal-based behavioural tasks (Novel object recognition (NOR) and Object Place Recognition (OPR)), prevention of the loss in dendritic spine density, reduction of neuroinflammation, normalization of LTP changes.

A proper control is a healthy subject or a subject not affected by AD or a subject not affected by a non-AD tauopathy.

A preferred embodiment of such a humanized antibody is a humanized 12A12 antibody or an epitope-binding fragment thereof.

In more preferred embodiments, there are provided resurfaced or humanized versions of the 12A12 antibody wherein surface-exposed residues of the antibody or its fragments are replaced in both light and heavy chains to more closely resemble known human antibody surfaces. The humanized 12A12 antibody or epitope-binding fragments thereof of the present invention have improved properties. For example, humanized 12A12 antibody or epitope-binding fragments thereof specifically recognizes an antigen comprising the sequence QGGYTMHQDQ (SEQ ID No. 1). More preferably, the humanized antibodies or epitope-binding fragments thereof have the additional ability to possesses at least one biological activity selected from inhibition of pathological hyperphosphorylation of Tau, reduction of amyloid precursor protein (APP)-derived Aβ species (monomer and low-molecular weight oligomers), increase in task-induced Arc expression when compared to a proper control, significant neuroprotection in at least one of two different hippocampal-based behavioural tasks (Novel object recognition (NOR) and Object Place Recognition (OPR)), prevention of the loss in dendritic spine density, reduction of neuroinflammation, normalization of LTP changes.

Nucleic acids encoding the antibodies of the invention are provided. In one embodiment, the nucleic acid molecule encodes a heavy and/or a light chain of an antibody of the invention. In a preferred embodiment, a single nucleic acid encodes a heavy chain of an anti-QGGYTMHQDQ (SEQ ID No. 1) immunoglobulin and another nucleic acid molecule encodes the light chain of an anti-QGGYTMHQDQ (SEQ ID No. 1) immunoglobulin.

The invention provides vectors comprising the polynucleotides of the invention. In one embodiment, the vector contains a polynucleotide encoding a heavy chain of an anti-QGGYTMHQDQ (SEQ ID No. 1) immunoglobulin. In another embodiment, said polynucleotide encodes the light chain of an anti-QGGYTMHQDQ (SEQ ID No. 1) immunoglobulin. The invention also provides vectors comprising polynucleotide molecules encoding, fusion proteins, modified antibodies, antibody fragments, and probes thereof.

In order to express the heavy and/or light chain of the antibodies of the invention, the polynucleotides encoding said heavy and/or light chains are inserted into expression vectors such that the genes are operatively linked to transcriptional and translational sequences.

Expression vectors include plasmids, YACs, cosmids, retrovirus, EBV-derived episomes, and all the other vectors that the skilled man will know to be convenient for ensuring the expression of said heavy and/or light chains. The skilled man will realize that the polynucleotides encoding the heavy and the light chains can be cloned into different vectors or in the same vector. In a preferred embodiment, said polynucleotides are cloned in the same vector.

Polynucleotides of the invention and vectors comprising these molecules can be used for the transformation of a suitable mammalian host cell. Transformation can be by any known method for introducing polynucleotides into a cell host. Such methods are well known of the man skilled in the art and include dextran-mediated transformation, calcium phosphate precipitation, polybrene-mediated transfection, protoplast fusion, electroporation, encapsulation of the polynucleotide into liposomes, biolistic injection and direct microinjection of DNA into nuclei.

The antibodies of the present invention include both the full-length antibodies discussed above, as well as epitope-binding fragments thereof. As used herein, “antibody fragments” include any portion of an antibody that retains the ability to bind to the epitope recognized by the full-length antibody, generally termed “epitope-binding fragments.” Examples of antibody fragments include, but are not limited to, Fab, Fab′ and F(ab′)2, Fd, single-chain Fvs (scFv), single-chain antibodies, disulfide-linked Fvs (dsFv) and fragments comprising either a VL or VH region. Epitope-binding fragments, including single-chain antibodies, may comprise the variable region(s) alone or in combination with the entirety or a portion of the following: hinge region, CH1, CH2, and CH3 domains.

Such fragments may contain one or both Fab fragments or the F(ab′)2 fragment. Preferably, the antibody fragments contain all six CDRs of the whole antibody, although fragments containing fewer than all of such regions, such as three, four or five CDRs, are also functional. Further, the fragments may be or may combine members of any one of the following immunoglobulin classes: IgG, IgM, IgA, IgD, or IgE, and the subclasses thereof.

Fab and F(ab′)2 fragments may be produced by proteolytic cleavage, using enzymes such as papain (Fab fragments) or pepsin (F(ab′)2 fragments).

The “single-chain FVs” (“scFvs”) fragments are epitope-binding fragments that contain at least one fragment of an antibody heavy chain variable region (VH) linked to at least one fragment of an antibody light chain variable region (VL). The linker may be a short, flexible peptide selected to assure that the proper three-dimensional folding of the VL and VH regions occurs once they are linked so as to maintain the target molecule binding-specificity of the whole antibody from which the single-chain antibody fragment is derived. The carboxyl terminus of the VL or VH sequence may be covalently linked by a linker to the amino acid terminus of a complementary VL or VH sequence.

Single-chain antibody fragments of the present invention contain amino acid sequences having at least one of the variable or complementarity determining regions (CDRs) of the whole antibodies described in this specification, but lack some or all of the constant domains of those antibodies. These constant domains are not necessary for antigen binding but constitute a major portion of the structure of whole antibodies. Single-chain antibody fragments may therefore overcome some of the problems associated with the use of antibodies containing a part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely to provoke an immune response in a recipient than whole antibodies.

Single-chain antibody fragments may be generated by molecular cloning, antibody phage display library or similar techniques well known to the skilled artisan. These proteins may be produced, for example, in eukaryotic cells or prokaryotic cells, including bacteria. The epitope-binding fragments of the present invention can also be generated using various phage display methods known in the art. In phage display methods, functional antibody domains are displayed on the surface of phage particles which carry the polynucleotide sequences encoding them. In particular, such phage can be utilized to display epitope-binding domains expressed from a repertoire or combinatorial antibody library (e.g., human or murine). Phage expressing an epitope-binding domain that binds the antigen of interest can be selected or identified with antigen, e.g., using labeled antigen bound or captured to a solid surface or bead. Phage used in these methods are typically filamentous phage including fd and M13 binding domains expressed from phage with Fab, Fv or disulfide-stabilized Fv antibody domains recombinantly fused to either the phage gene III or gene VIII protein.

Examples of phage display methods that can be used to make the epitope-binding fragments of the present invention include those disclosed in Brinkman et al., 1995, J. Immunol. Methods, 182: 41-50; Ames et al., 1995, J. Immunol. Methods, 184: 177-186; Kettleborough et al., 1994, Eur. J. Immunol., 24: 952-958; Persic et al., 1997, Gene, 187: 9-18; Burton et al., 1994, Advances in Immunology, 57: 191-280; WO/1992/001047; WO 90/02809; WO 91/10737; WO 92/01047; WO 92/18619; WO93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

After phage selection, the regions of the phage encoding the fragments can be isolated and used to generate the epitope-binding fragments through expression in a chosen host, including mammalian cells, insect cells, plant cells, yeast, and bacteria, using recombinant DNA technology, e.g., as described in detail below. For example, techniques to recombinantly produce Fab, Fab′ and F(ab′)2 fragments can also be employed using methods known in the art such as those disclosed in WO 92/22324; Mullinax et al., 1992, BioTechniques, 12(6): 864-869; Sawai et al., 1995, AJRI, 34: 26-34; and Better et al., 1988, Science, 240:1041-1043; said references incorporated by reference in their entireties. Examples of techniques which can be used to produce single-chain Fvs and antibodies include those described in U.S. Pat. Nos. 4,946,778 and 5,258,498; Huston et al., 1991, Methods in Enzymology, 203: 46-88; Shu et al., 1993, PNAS, 90: 7995-7999; Skerra et al., 1988, Science, 240:1038-1040.

Also included within the scope of the invention are functional equivalents of the antibody of the invention and the humanized antibody of the invention. The term “functional equivalents” includes antibodies with homologous sequences, chimeric antibodies, artificial antibodies and modified antibodies, for example, wherein each functional equivalent is defined by its ability to bind to an antigen comprising the sequence QGGYTMHQDQ (SEQ ID No. 1). The skilled artisan will understand that there is an overlap in the group of molecules termed “antibody fragments” and the group termed “functional equivalents.” Methods of producing functional equivalents are known to the person skilled in the art and are disclosed, for example, in WO 93/21319, EP 239,400; WO 89/09622; EP 338,745; and EP 332,424, which are incorporated in their respective entireties by reference.

Antibodies with homologous sequences are those antibodies with amino acid sequences that have sequence homology with amino acid sequence of 12A12 antibody and a humanized 12A12 antibody of the present invention. Preferably homology is with the amino acid sequence of the variable regions of the 12A12 antibody and humanized 12A12 antibody of the present invention. “Sequence homology” as applied to an amino acid sequence herein is defined as a sequence with at least about 90%, 91%, 92%, 93%, or 94% sequence homology, and more preferably at least about 95%, 96%, 97%, 98%, or 99% sequence homology to another amino acid sequence, as determined, for example, by the FASTA search method in accordance with Pearson and Lipman, 1988, Proc. Natl. Acad. ScL USA, 85: 2444-2448.

Artificial antibodies include scFv fragments, diabodies, triabodies, tetrabodies and mm (see reviews by Winter, G. and Milstein, C, 1991, Nature, 349: 293-299; Hudson, P J., 1999, Current Opinion in Immunology, 11: 548-557), each of which has antigen-binding ability. In the single chain Fv fragment (scFv), the VH and VL domains of an antibody are linked by a flexible peptide. Typically, this linker peptide is about 15 amino acid residues long. If the linker is much smaller, for example 5 amino acids, diabodies are formed, which are bivalent scFv dimers. If the linker is reduced to less than three amino acid residues, trimeric and tetrameric structures are formed that are called triabodies and tetrabodies. The smallest binding unit of an antibody is a CDR, typically the CDR2 of the heavy chain which has sufficient specific recognition and binding that it can be used separately. Such a fragment is called a molecular recognition unit or mru. Several such mrus can be linked together with short linker peptides, therefore forming an artificial binding protein with higher avidity than a single mru.

The functional equivalents of the present application also include modified antibodies, e.g., antibodies modified by the covalent attachment of any type of molecule to the antibody. For example, modified antibodies include antibodies that have been modified, e.g., by glycosylation, acetylation, pegylation, phosphorylation, amidation, derealization by known protecting/blocking groups, proteolytic cleavage, linkage to a cellular ligand or other protein, etc. The covalent attachment does not prevent the antibody from generating an anti-idiotypic response. These modifications may be carried out by known techniques, including, but not limited to, specific chemical cleavage, acetylation, formylation, metabolic synthesis of tunicamycin, etc. Additionally, the modified antibodies may contain one or more non-classical amino acids.

Functional equivalents may be produced by interchanging different CDRs on different chains within different frameworks. Thus, for example, different classes of antibody are possible for a given set of CDRs by substitution of different heavy chains, whereby, for example, lgG1-4, IgM1 lgA1-2, IgD, IgE antibody types and isotypes may be produced. Similarly, artificial antibodies within the scope of the invention may be produced by embedding a given set of CDRs within an entirely synthetic framework.

Functional equivalents may be readily produced by mutation, deletion and/or insertion within the variable and/or constant region sequences that flank a particular set of CDRs, using a wide variety of methods known in the art. The antibody fragments and functional equivalents of the present invention encompass those molecules with a detectable degree of binding to an antigen comprising the sequence QGGYTMHQDQ (SEQ ID No. 1). A detectable degree of binding includes all values in the range of at least 10-100%, preferably at least 50%, 60% or 70%, more preferably at least 75%, 80%, 85%, 90%, 95% or 99% of the binding ability of the murine 12A12 antibody to an antigen comprising QGGYTMHQDQ (SEQ ID No. 1).

The CDRs are of primary importance for epitope recognition and antibody binding. However, changes may be made to the residues that comprise the CDRs without interfering with the ability of the antibody to recognize and bind its cognate epitope. For example, changes that do not affect epitope recognition, yet increase the binding affinity of the antibody for the epitope may be made.

Thus, also included in the scope of the present invention are improved versions of both the murine and humanized antibodies, which also specifically recognize and bind an antigen comprising the sequence QGGYTMHQDQ (SEQ ID No. 1), preferably with increased affinity.

Several studies have surveyed the effects of introducing one or more amino acid changes at various positions in the sequence of an antibody, based on the knowledge of the primary antibody sequence, on its properties such as binding and level of expression (Yang, W. P. et al., 1995, J. MoI. Biol., 254: 392-403; Rader, C. et al., 1998, Proc. Natl. Acad. Sci. USA, 95: 8910-8915; Vaughan, T. J. ef al., 1998, Nature Biotechnology, 16: 535-539).

In these studies, equivalents of the primary antibody have been generated by changing the sequences of the heavy and light chain genes in the CDR1, CDR2, CDR3, or framework regions, using methods such as oligonucleotide-mediated site-directed mutagenesis, cassette mutagenesis, error-prone PCR, DNA shuffling, or mutator-strains of E. coli (Vaughan, T. J. ef al., 1998, Nature Biotechnology, 16: 535-539; Adey, N. B. et al., 1996, Chapter 16, pp. 277-291, in “Phage Display of Peptides and Proteins”, Eds. Kay, B. K. et al., Academic Press). These methods of changing the sequence of the primary antibody have resulted in improved affinities of the secondary antibodies (Gram, H. et al., 1992, Proc. Natl. Acad. Sci. USA, 89: 3576-3580; Boder, E. T. ef al., 2000, Proc. Natl. Acad. Sci. USA, 97: 10701-10705; Davies, J. and Riechmann, L., 1996, Immunotechnolgy, 2: 169-179; Thompson, J. ef al., 1996, J. MoI. Biol., 256: 77-88; Short, M. K. ef al., 2002, J. Biol. Chem., 277: 16365-16370; Furukawa, K. et al., 2001, J. Biol. Chem., 276: 27622-27628).

By a similar directed strategy of changing one or more amino acid residues of the antibody, the antibody sequences described in this invention can be used to develop antibodies with improved functions, including improved affinity for an antigen comprising the sequence QGGYTMHQDQ (SEQ ID No. 1).

Preferred amino acid substitutions are those which: (1) reduce susceptibility to proteolysis, (2) reduce susceptibility to oxidation, (3) alter binding affinity for forming protein complexes, and (4) confer or modify other physico-chemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain (s) forming intermolecular contacts. A conservative amino acid substitution should not substantially change the structural characteristics of the parent sequence (e.g., a replacement amino acid should not tend to break a helix that occurs in the parent sequence or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N. Y. (1991)); and Thornton et al., 1991, Nature, 354: 105, which are each incorporated herein by reference.

Improved antibodies also include those antibodies having improved characteristics that are prepared by the standard techniques of animal immunization, hybridoma formation and selection for antibodies with specific characteristics.

Improved antibodies according to the invention include in particular antibodies with enhanced functional properties. It is also possible to use cell lines specifically engineered for production of improved antibodies. In particular, these lines have altered regulation of the glycosylation pathway, resulting in antibodies which are poorly fucosylated or even totally defucosylated. Such cell lines and methods for engineering them are disclosed in e.g. Shinkawa et al. (2003, J. Biol. Chem. 278(5): 3466-3473), Ferrara et al. (2006, J. Biol. Chem. 281(8): 5032-5036; 2006, Biotechnol. Bioeng. 93(5): 851-61), EP 1331266, EP 1498490, EP 1498491, EP 1676910, EP 1792987, and WO 99/54342.

The present invention also includes conjugates. These conjugates comprise two primary components, a cell-binding agent and a therapeutic agent.

The invention also relates to a therapeutic composition for the treatment and/or prevention of Alzheimer's disease or a non-AD tauopathy.

The instant invention provides pharmaceutical compositions comprising:

a) an effective amount of an antibody, antibody fragment or antibody conjugate of the present invention, and;

b) a pharmaceutically acceptable carrier, which may be inert or physiologically active.

As used herein, “pharmaceutically-acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH ˜7.4, containing or not containing about 1 mg/ml to 25 mg/ml human serum albumin, (2) 0.9% saline (0.9% w/v sodium chloride (NaCl)), and (3) 5% (w/v) dextrose; and may also contain an antioxidant such as tryptamine and a stabilizing agent such as Tween 20.

The compositions of the invention may be in a variety of forms. These include for example liquid, semi-solid, and solid dosage forms, but the preferred form depends on the intended mode of administration and therapeutic application. Typical preferred compositions are in the form of injectable or infusible solutions. The preferred mode of administration is parenteral (e.g. intravenous, intramuscular, intraperinoneal, subcutaneous). In a preferred embodiment, the compositions of the invention are administered intravenously as a bolus or by continuous infusion over a period of time. In another preferred embodiment, they are injected by intramuscular, subcutaneous, intra-articular, intrasynovial, intratumoral, peritumoral, intralesional, or perilesional routes, to exert local as well as systemic therapeutic effects.

Sterile compositions for parenteral administration can be prepared by incorporating the antibody, antibody fragment or antibody conjugate of the present invention in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohols, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

The antibody, antibody fragment or antibody conjugate of the present invention may also be orally administered. As solid compositions for oral administration, tablets, pills, powders (gelatine capsules, sachets) or granules may be used. In these compositions, the active ingredient according to the invention is mixed with one or more inert diluents, such as starch, cellulose, sucrose, lactose or silica, under an argon stream. These compositions may also comprise substances other than diluents, for example one or more lubricants such as magnesium stearate or talc, a coloring, a coating (sugar-coated tablet) or a glaze.

As liquid compositions for oral administration, there may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The doses depend on the desired effect, the duration of the treatment and the route of administration used; they are generally between 5 mg and 1000 mg per day orally for an adult with unit doses ranging from 1 mg to 250 mg of active substance. In general, the doctor will determine the appropriate dosage depending on the age, weight and any other factors specific to the subject to be treated.

Also included are resurfaced or humanized versions of the 12A12 antibody wherein surface-exposed residues of the variable region frameworks of the antibodies, or their epitope-binding fragments, are replaced in both light and heavy chains to more closely resemble known human antibody surfaces. The humanized antibodies and epitope-binding fragments thereof of the present invention have improved properties in that they are less immunogenic (or completely non-immunogenic) than murine versions in human subjects to which they are administered.

Thus, the different versions of humanized 12A12 antibody and epitope-binding fragments thereof of the present invention specifically recognize an antigen comprising the sequence QGGYTMHQDQ (SEQ ID No. 1) while not being immunogenic to a human.

The present invention also includes kits, e.g., comprising the described antibody and/or conjugate and instructions for the use of the antibody and/or conjugate for treating the present pathologies. The instructions may include directions for using the antibody and/or conjugate in vitro, in vivo or ex vivo.

Typically, the kit will have a compartment containing the antibody and/or conjugate. The antibody and/or conjugate may be in a lyophilized form, liquid form, or other form amendable to being included in a kit. The kit may also contain additional elements needed to practice the method described on the instructions in the kit, such a sterilized solution for reconstituting a lyophilized powder, additional agents for combining with the antibody and/or conjugate prior to administering to a patient, and tools that aid in administering the antibody and/or conjugate to a patient.

The kit may also include components necessary for the preparation of a pharmaceutically acceptable formulation, such as a diluent if the antibody and/or conjugate is in a lyophilized state or concentrated form, and for the administration of the formulation.

It is also an object of the invention a method of treating and/or preventing AD or a non-AD tauopathy comprising administering a therapeutically effective amount the antibody or fragment or derivative or conjugate thereof as above defined.

Included in the present invention are also nucleic (or amino) acid sequences derived from the nucleotide (or amino acid) sequences shown below, e.g. functional fragments, mutants, derivatives, analogues, and sequences having a % of identity of at least 70% with the below sequences.

The present invention will be illustrated by means of non-limiting examples in reference to the following figures.

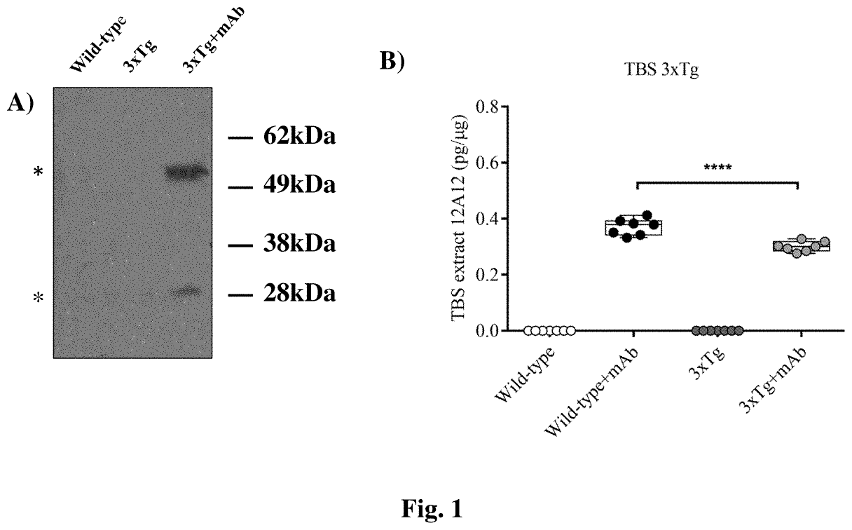

FIG. 1 i.v.-delivered 12A12mAb is able to cross the BBB and get access into hippocampus: the injected 12A12mAbanti-tau antibody is present and biologically active in the brain from immunized animals.

A) Western blotting analysis was carried out by probing with anti-mouse IgG as primary antibody on hippocampal protein extracts from animals of the three experimental groups (wild-type, naive Tg-AD, Tg-AD+mAb) which underwent i.v. injection with saline or 12A12mAb (see details in methods). Notice that 3XTg animals which were systemically i.v. injected for 14 days with 12A12mAb, exhibited high levels of cerebral mouse IgG when compared to not-vaccinated controls.

B) Levels of anti-tau antibody 12A12mAb were evaluated by enzyme-linked immunosorbent assay (ELISA) in the TBS extracts from wild-type and 3xTg-AD mice that i.v. received 12A12mAb for 14 days (see details in methods). The ELISA used to measure the anti-tau antibody relies on the plate-immobilized recombinant NH₂26-44 tau, such that free antibody can readily bind to immobilized tau and be detected, whereas antibody already bound to tau will not be detected. Notice that a significant portion of the 12A12mAb in 3xTg-AD brains is bound to endogenous NH₂htau and does nonspecifically interact with the large amount of intracellular tau released during homogenization. Statistically significant differences (see details in the main text) were calculated by analysis of variance (ANOVA) followed post-hoc test for multiple comparison among more than two groups. p<0.05 was accepted as statistically significant.

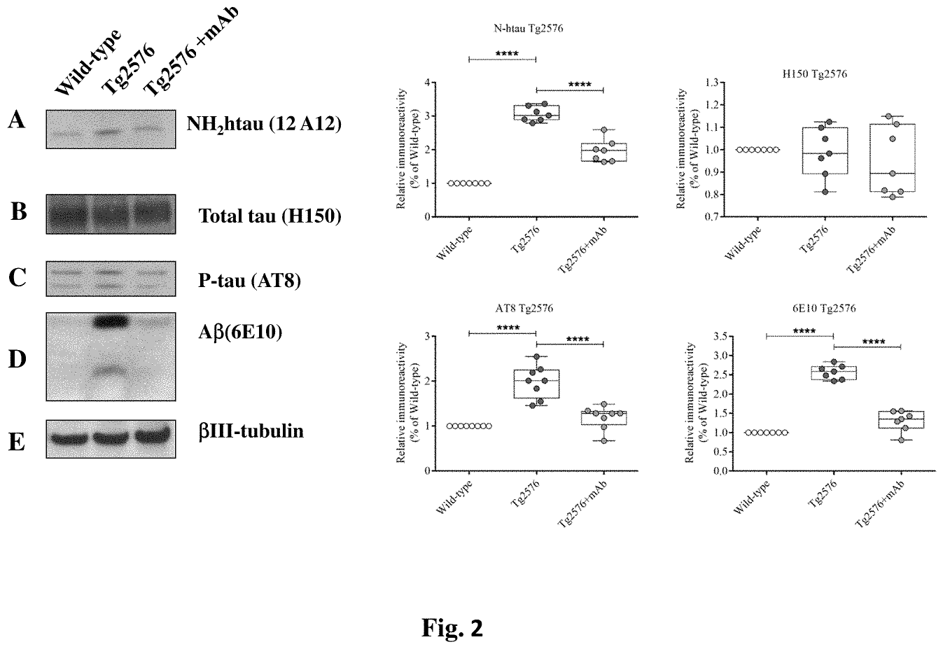

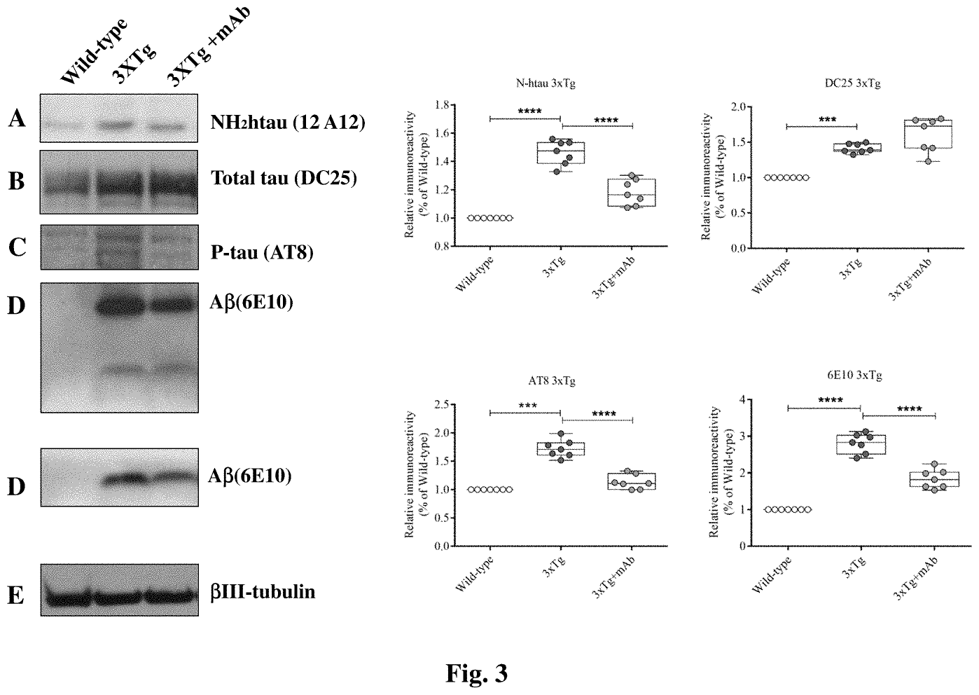

FIG. 2-3 Reduction of the NH₂htau in Tg-AD mice immunized with 12A12mAb ameliorates the disease-associated synaptic neuropathology.

Representative blots (n=5) of SDS-PAGE Western blotting analysis (right) on isolated synaptosomal preparations from hippocampal region of animals from three experimental groups (wild-type, Tg-AD and Tg-AD+mAb) of both strains (Tg2576, 3xTg) to assess the content of the NH₂htau fragment (A), total tau full-length (B), AT8-phosphorylated tau(C), Aβ monomers and oligomeric species (D). β-III tubulin was used as loading control (E) and relative densitometric quantifications were reported (left). Data are presented as the mean (±SEM) and statistically significant differences (see details in the main text) were calculated by analysis of variance (ANOVA) followed by post-hoc test for multiple comparison among more than two groups. p<0.05 was accepted as statistically significant (*p<0.05; **p<0.01; ***p<0.0005; *** p<0.0001).

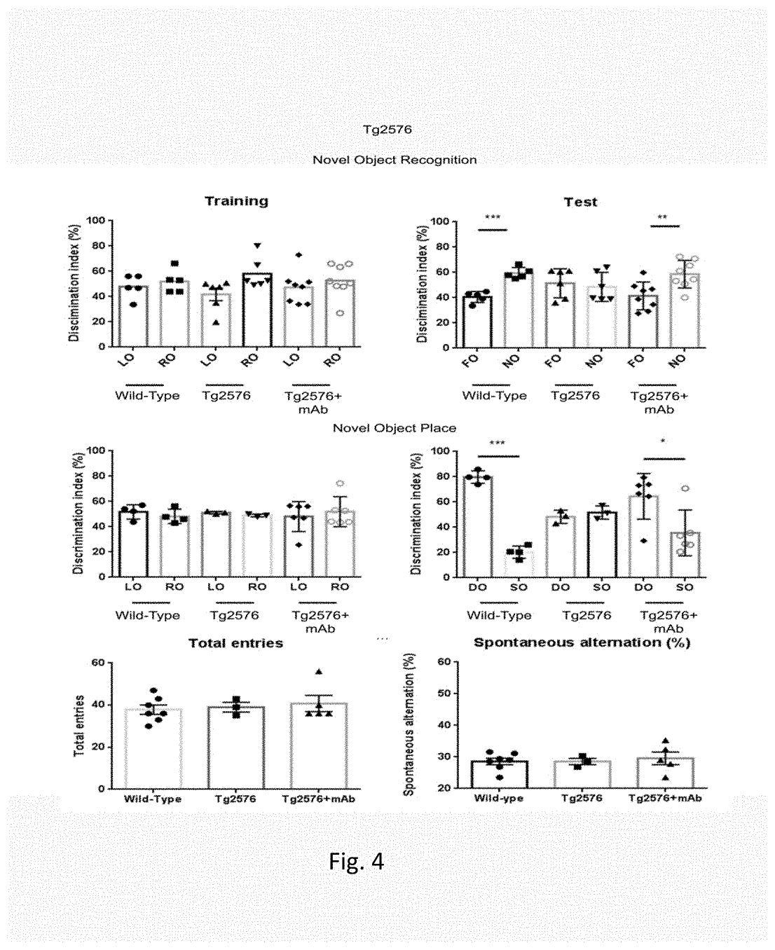

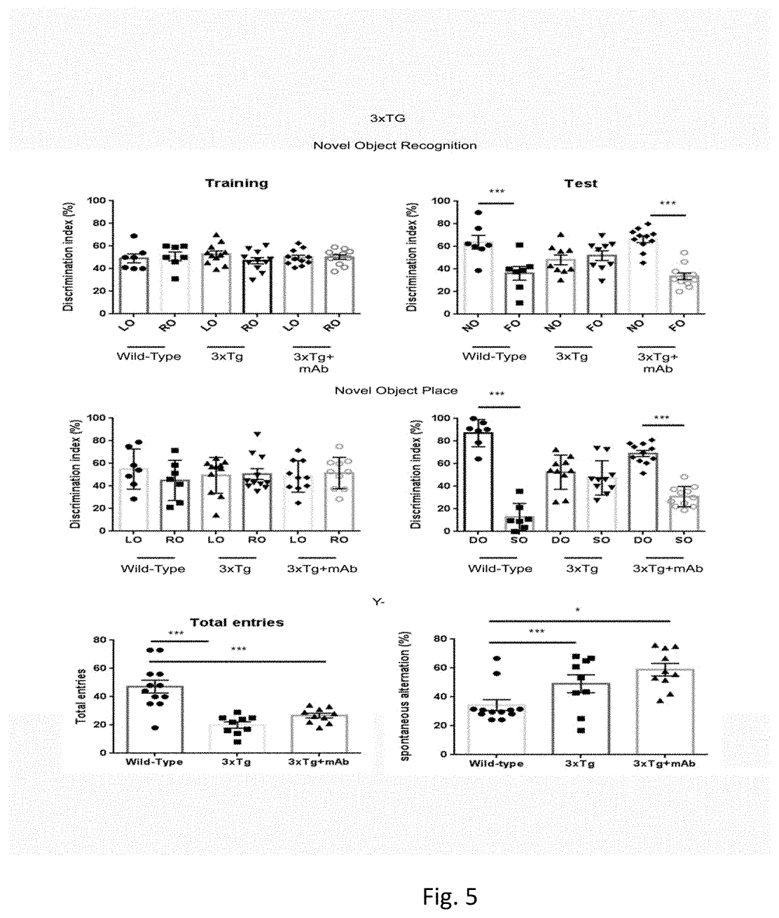

FIG. 4-5 Improved cognition in Tg ADmice immunized with 12A12mAb.

14 days days after i.v. 12A12mAb immunization, the in vivo effect of NH₂htau on cognitive performance was investigated in animals from the three experimental groups (wild-type, Tg-AD and Tg-AD+mAb) of both genetic backgrounds (Tg2576, 3xTg) in the novel object recognition (NOR), novel object place (NOP) and Y-maze (top to bottom) tasks. For NOR and NOP: Right and left histograms respectively represent the total time (s) spent to explore the object during training and the discrimination/preference index (%) of corresponding values measured during the test trial among animals from the different experimental groups (wild-type, Tg-AD and Tg-AD+mAb) of both genetic backgrounds (Tg2576, 3xTg). The columns color refers to objects presented during training (LO=left object; RO=right object) and test-trial (FO=familiar object; NO=novel object; DO=displaced object; SO=stationary object). Analysis of preference index (%) measured as time spending in the exploration of the novel/displaced object/(time spending in the exploration of novel/displaced object+time spending in the exploration of familiar/stationary object)×100. Data were expressed as means±S.E.M. (n=6-10). Values are means of at least three independent experiments and statistically significant differences (see details in the main text) were calculated by analysis of variance (ANOVA) followed by post-hoc test for multiple comparison among more than two groups. p<0.05 was accepted as statistically significant (*p<0.05; **p<0.01; ***p<0.0005; *** p<0.0001). For Y-maze: Right and left histograms, respectively, represent the total entries (the total arm entries correspond to the total number of arms entered) and the spontaneous alternation (the number of alternations corresponds to the successive entries into 3 different arms in overlapping triplet sets) of animals from the different experimental groups (wild-type, Tg-AD and Tg-AD+mAb) of both genetic backgrounds (Tg2576, 3xTg). The percentage alternation was calculated as the ratio between number of correct triplets (e.g. ABC) and total entrances minus 2, multiplied by 100. Values are means of at least three independent experiments and statistically significant differences (see details in the main text) were calculated by analysis of variance (ANOVA) followed by post-hoc test for multiple comparison among more than two groups. p<0.05 was accepted as statistically significant (*p<0.05; **p<0.01; ***p<0.0005; *** p<0.0001).

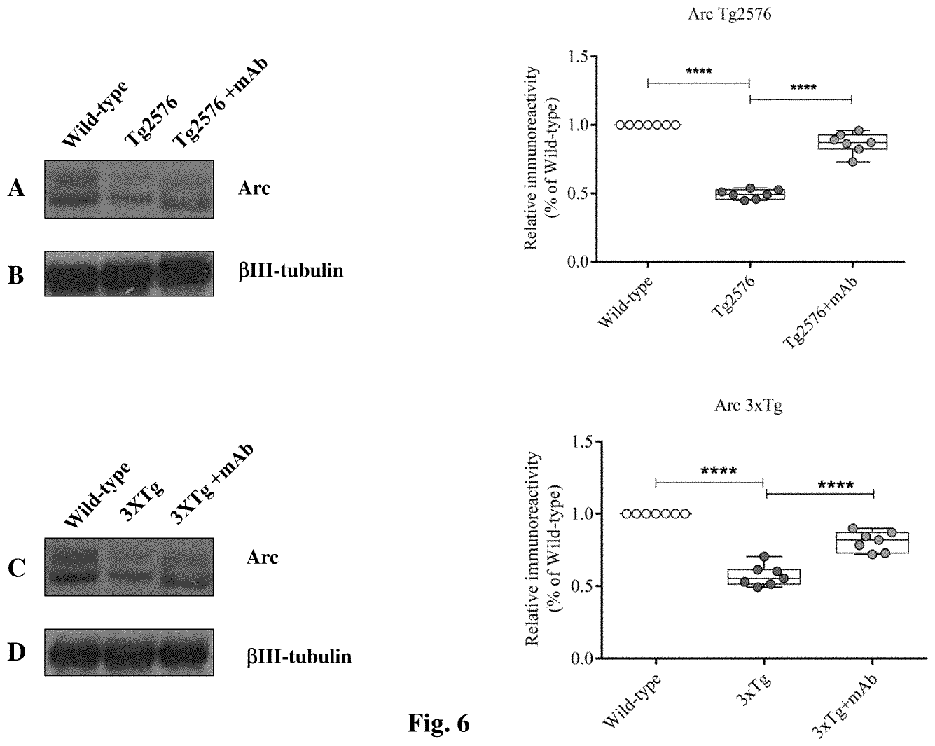

FIG. 6 The activity-regulated cytoskeleton-associated protein Arc is upregulated in synapses from 12A12mAb-vaccinated Tg-AD mice of both genetic background.

Representative blots (n=4) of SDS-PAGE Western blotting analysis (left) on isolated synaptosomal preparations from hippocampal region of animals from three experimental groups (wild-type, Tg-AD and Tg-AD+mAb) of both strains (Tg2576, 3xTg) sacrified at the end of test session to assess the content of the activity-regulated cytoskeleton-associated protein Arc (A-C) which is normally evoked by short-term memory/learning task. β-III tubulin (B-D) was used as loading control and relative densitometric quantifications were reported (right). Data are presented as the mean (±SEM) and statistically significant differences (see details in the main text) were calculated by analysis of variance (ANOVA) followed by post-hoc test for multiple comparison among more than two groups. p<0.05 was accepted as statistically significant (*p<0.05; **p<0.01; ***p<0.0005; *** p<0.0001).

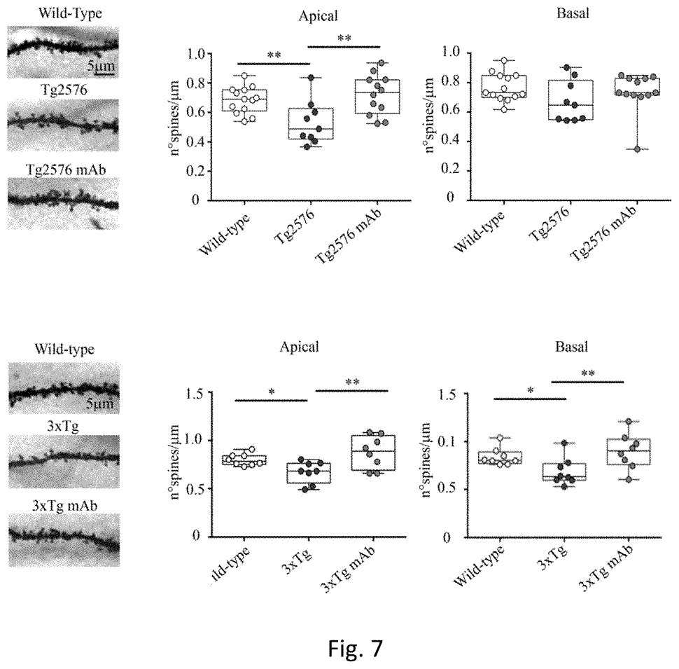

FIG. 7 Immunization with 12A12mAb in Tg-AD mice is protective against the dendritic spines density loss which affects the memory and learning processes.

Comparative photomicrographs of Golgi-stained hippocampal CA1 neurons showing dendritic segments from animals from three experimental groups (wild-type, Tg-AD and Tg-AD+mAb) of both strains (Tg2576, 3xTg) (left, refers to CA1 pyramidal neurons dendrites scale bar 5 μm). Histograms (right) depict the morphometric analysis of the dendritic spine density from the three experimental groups (at least 15 neurons from three mice for experimental group were used for quantitative analysis). Values are expressed as number of spines (mean±S.E.M) per 1 μm segment. Statistically significant differences (see details in the main text) were calculated by analysis of variance (ANOVA) followed by post-hoc test for multiple comparison among more than two groups. p<0.05 was accepted as statistically significant (*p<0.05; **p<0.01; ***p<0.0005; *** p<0.0001).

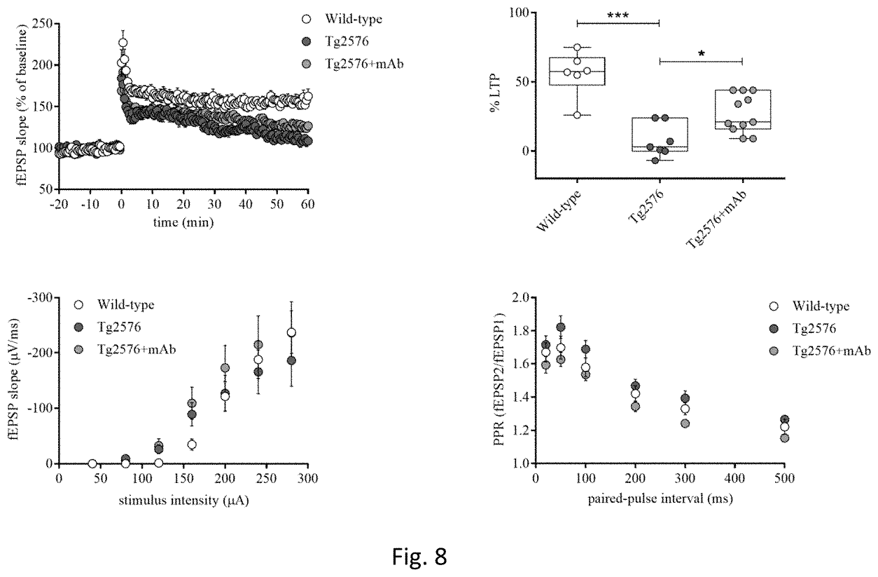

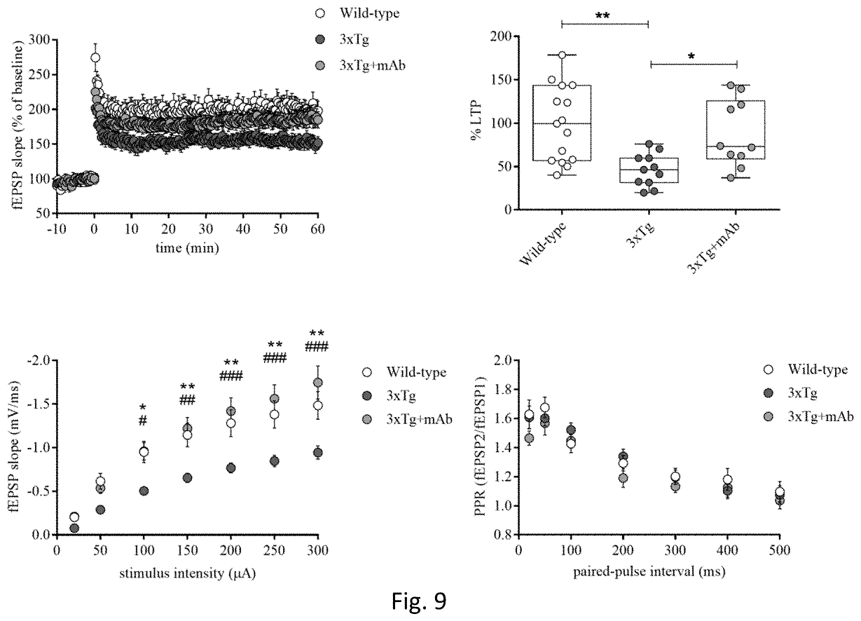

FIG. 8-9 Reduction of cognitive deficits in 12A12mAb-immunized Tg-AD mice correlates with an increased LTP.

Top): Time plot of average fEPSP responses and changes in magnitude of CA1-LTP were calculated among animals (n=6-10) from three experimental groups (wild-type, Tg-AD and Tg-AD+mAb) of both strains (Tg2576, 3xTg). At least 7 slices from 6 different mice were recorded for each experimental condition. Data are presented as the mean (±SEM) and statistically significant differences (see details in the main text) were calculated by analysis of variance (ANOVA) followed by post-hoc test for multiple comparison among more than two groups. p<0.05 was accepted as statistically significant (*p<0.05; **p<0.01; ***p<0.0005; *** p<0.0001).

Bottom): Input/output curves as plots of the fEPSP slopes against the corresponding stimulus intensity (right) were calculated from hippocampal slices of animals (n=6-10) from three experimental groups (wild-type, Tg-AD and Tg-AD+mAb) of both strains (Tg2576, 3xTg). Comparison of paired-pulse facilitation (PPF) in animals (n=6-10) from three experimental groups (wild-type, Tg-AD and Tg-AD+mAb) of both strains (Tg2576, 3xTg) was also shown (left). PPF was induced by pairs of stimuli delivered at increasing interpulse intervals (20, 50, 100, 200, 500 msec). Data are presented as the mean (±SEM) facilitation ratio of the second response relative to the first response. Statistically significant differences (see details in the main text) were calculated by analysis of variance (ANOVA) followed by post-hoc test for multiple comparison among more than two groups. p<0.05 was accepted as statistically significant. (*p<0.05; **p<0.01; ***p<0.0005; *** p<0.0001).

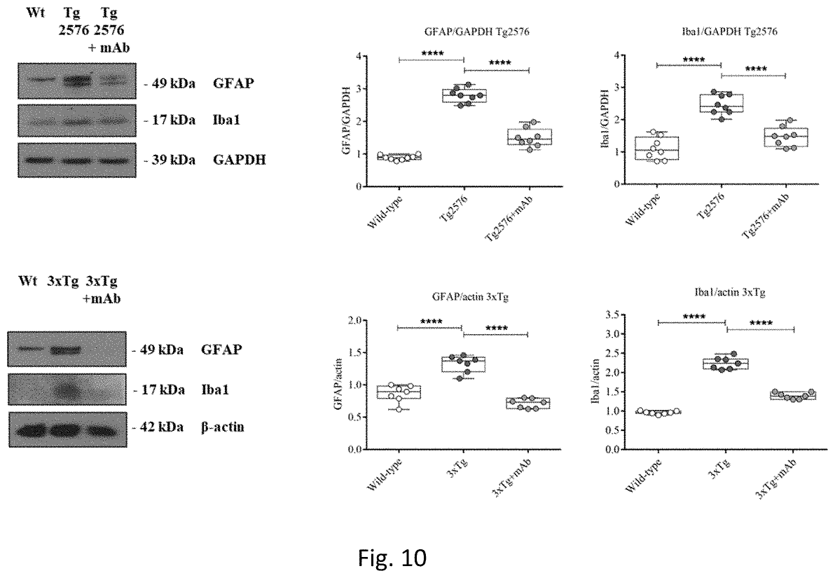

FIG. 10 Inflammatory response (activation of astrocytes and microglia) is strongly downregulated in 12A12mAb-immunized Tg-AD mice.

Neuroinflammation processes (activation of astrocytes and microglia) was assessed on hippocampal extracts from animals from three experimental groups (wild-type, Tg-AD and Tg-AD+mAb) of both strains (Tg2576, 3xTg) by Western blotting analysis (right) for inflammatory proteins (GFAP, Iba1, respectively). Relative densitometric quantification of intensity signals (left) indicates lower levels of GFAP and Iba1 in Tg-AD mice+mAb compared to not-immunized Tg-AD. GAPDH (glyceraldehyde 3-phosphate dehydrogenase) serves as loading control. Values are means of at least three independent experiments and statistically significant differences (see details in the main text) were calculated by analysis of variance (ANOVA) followed by post-hoc test for multiple comparison among more than two groups. p<0.05 was accepted as statistically significant (*p<0.05; **p<0.01; ***p<0.0005;* p<0.0001).

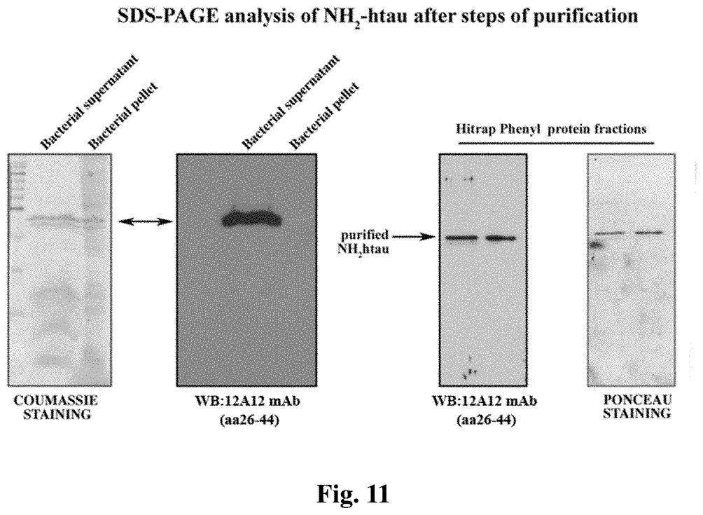

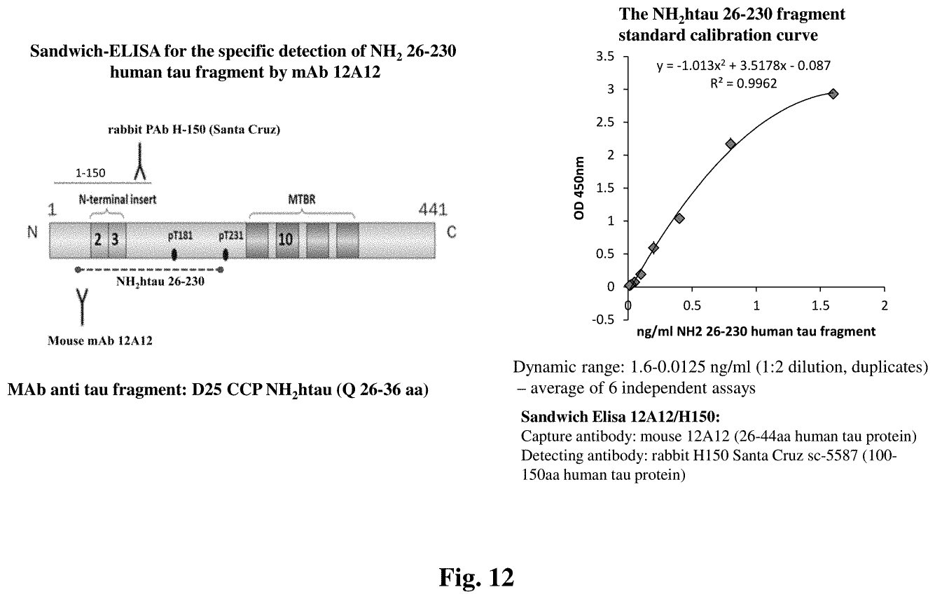

FIG. 11-12 12A12mAb binds the recombinant, purified the NH2 26-230 tau fragment.

SDS-PAGE analysis probing with 12A12mAb was carried out to check its ability of binding the recombinant, purified the NH₂ 26-230 tau fragment. The NH₂htau 26-230 fragment calibration curve was calculated by sandwich Enzyme-linked immunosorbent assay (ELISA) 12A12/H150 using as
Capture antibody: mouse 12A12 (26-44aa human tau protein)
Detecting antibody: rabbit H150 Santa Cruz sc-5587 (100-150aa human tau protein).

### DETAILED DESCRIPTION OF THE INVENTION

Materials and Methods

Animals

All protocols involving animals were performed in accordance with the guidelines established by the European Communities Council (Directive 2010/63/EU of 22 Sep. 2010). Experiments involving animals were performed in accordance with the relevant approved guidelines and regulations accepted by the Italian Ministry of Health and approved by the Ethical Committee on animal experiments of EBRI “Rita Levi-Montalcini” Foundation (Rome, Italy). Male mice overexpressing the APP695 fragment with the Swedish mutation (TgHuAPP695swe: Tg2576) in a hybrid genetic background (87% C57BL/6×12.5% SJL) were subsequently backcrossed to C57BL/6×SJL F1 females. Tg2576 transgenic mice, created in the laboratory of Dr. Hsiao (Hsiao et al., 1996), were obtained from The Jackson Laboratory (MGI:2385631). The offspring was genotyped to confirm the presence of human mutant APP DNA sequence by PCR. Each experiment was carried out in transgenic mice and wild-type (WT) mice of 3, and 6 months of age. Mice were housed (four or five animals per cage) in pathogen-free facilities with 12-h light/12-h dark cycles with ad libitum access to food and water. The homozygous 3xTg mice harboring human APPSWE and tauP301L transgenes with knock-in PS1M146V under the control of the mouse Thy1.2 promoter, created in the laboratory of Dr. Frank LaFerla (Oddo et al., 2003), were obtained from The Jackson Laboratory (https://www.jax.org/strain/004807) and bred on the mixed C7BL/6; 129X1/SvJ; 129S1/Sv genetic background. B6129SF2/J strain mice, used as wild-type (WT) controls in the present study, were the offspring of a cross between C57BL/6J females (B6) and 129S1/SvImJ males (129S); they are commonly used as controls for genetically engineered strains generated with 129-derived embryonic stem cells and maintained on a mixed B6;129 background (https://www.jax.org/strain/101045).

Generation of the N-Terminal Tau 12A12 Antibody (26-36Aa)

Monoclonal 12A12 was generated by immunizing mice with a peptide of amino acids 26-36 of hT40 D25(NH2-QGGYTMHQDQ (SEQ ID No. 1) —COOH epitopes). Affinity-purified mouse monoclonal cleavage-site antibody directed against the extreme N-terminal 26-36 aa of human tau protein (D25-(QGGYTMHQDQ) (SEQ ID No. 1)) (mAb12A12) was produced, purified and characterized according to standard procedures. The specificity of this mAb (IgG isotype) was verified by Western blot analysis (FIG. 11) and enzyme-linked immunosorbent assay (ELISA) test (95% sensitivity and 90% specificity) (FIG. 12). Antibodies were tested for endotoxin using an LAL chromogenic kit (Pierce, Rockford, Ill.) and contained <1 U/mg of endotoxin.

Immunization Scheme

The 3- and 6-month-old Tg2576 and 3XTg male mice (n=12-14 animals per group) were given two intravenous injections of 30 μg/animal of 12A12 mAb within 7 days for two weeks. Age-matched, transgenic and WT mice—which were infused under the same experimental conditions with vehicle (saline) only—were referred as control not-immunized groups. For intravenous injections, mice were placed in a restrainer (Braintree Scientific), and an inch of the tail was shaved and placed in warm water to dilate veins. Mice were then injected via the lateral tail vein, returned to home cages, and kept under observation.

TBS Extract

Mouse hippocampal was homogenized in 5 volumes (wt/vol) Tris-buffered saline (TBS), pH 7.4, plus proteases inhibitor cocktail (Sigma P8340) and phosphatase inhibitor cocktail (Sigma Aldrich, Oakville, Ontario, Canada P5726/P2850) with 30 strokes of a glass Dounce tissue. The homogenate was centrifuged at 90,000 g at 4° C. for 1 hour. The entire supernatant (TBS extract) was removed to clean tube and stored at −20° C.

Cloning, Bacterial Expression and Purification of NH2Htau-26-230 (i.e. NH2Htau)

cDNA fragment coding for the aminoacids 26-230 of the isoform 4 of human tau protein (htau40) is cloned into the vector pET-11a (Novagen) suitable for the expression of recombinant proteins in BL21DE3 Gold E. coli cells. After induction with IPTG, recombinant protein in lysates from bacterial pellet are purified to homogeneity by a two-step procedure: step 1 is a HiCood Q Sepharose 16/10; step2 is Hitrap Phenyl 5 ml. Degree of protein purification is evaluated by Coomassie Brilliant Blue G-250 and checked by SDS-PAGE under reducing conditions by Western blotting (WB) with commercial human-specific NH2-tau antibody (DC39N1 45-73 aa) and with 12A12 mAb. The molecular identity of purified peptide fraction is finally checked by electrospray ionization mass spectrometry (ESI-MS). As control, full-length tau protein isoform containing 2 N-terminal inserts and 4 microtubule binding repeats (htau40) is also cloned in pET-11a vector, expressed in BL21DE3 Gold E. coli cells and purified according to Barghorn and Mandelkow 2002

Detection of 12 A12 mAb in Brain by ELISA

The concentration of i.v. delivered anti-tau 12 A12 mAb was measured in TBS brain extracts using a solid-phase ELISA on the plate-immobilized synthetic NH₂26-44 which, being the minimal AD-relevant (Borreca et al., 2018) active moiety of the parental longer NH₂ 26-230 (Amadoro et al., 2004, 2006), was used as catching peptide. Clear 96 well high-binding plates (Costar, Corning, N.Y.) were coated with synthetic NH₂26-44 (50 μL of 5 μg/mL in PBS per well) for 1 hour at 37° C. Wells were washed twice with PBS-Tw and blocked by incubation with 200 μL 1% (wt/vol) BSA in PBS-Tw for 2 hours. Wells were washed again and loaded with the standard curve prepared by making serial dilutions of mouse N-terminal tau 12A12 antibody (250-0.12 ng/ml) in coating buffer (0.05 M Carbonate-Bicarbonate, pH 9.6) (50 μl/well), the TBS extracts diluted 1/50, 1/10, 1/2, 1/1.3 in coating buffer (50 μl/well) or blanks (50 μL/well) overnight at 4° C. Thereafter, wells were washed twice and incubated 50 μl/well of mouse HRP secondary antibody for 1 h at 37° C. before being washed again 3 times with PBS-Tw and detected with TMB substrate (T0440; Sigma Aldrich, Oakville, Ontario, Canada,). Luminescence counts were measured using a Packard TopCount (PerkinElmer, MA). Log-transformed luminescence counts from individual samples were interpolated to concentration using a second-order polynomial fit to the respective standards (GraphPad Prism 5.00, GraphPad Software, San Diego).

Electrophysiological Recordings

Field electrophysiological recordings recordings were performed on hippocampal coronal slices (400-μm-thick) obtained from six-month-old Tg ADmice and age-matched wild-type using standard procedures (Podda et al., 2016; Nobili et al., 2017). In detail, mice were anesthetized by halothane inhalation and decapitated. The brain was rapidly removed and put in ice-cold cutting solution (in mM: 124 NaCl, 3.2 KCl, 1 NaH2PO4, 26 NaHCO₃, 2 MgCl2, 1 CaCl₂), 10 glucose, 2 sodium pyruvate, and 0.6 ascorbic acid, bubbled with 95% O2-5% CO2; pH 7.4). Slices were cut with a vibratome (LEICA VT1200S) and incubated in artificial cerebrospinal fluid (aCSF; in mM: 124 NaCl; 3.2 KCl; 1 NaH2PO4, 1 MgCl2, 2 CaCl₂); 26 NaHCO₃; 10 glucose; pH 7.4; 95% O2-5% CO2) at 32° C. for 60 min and then at RT until use. Slices were transferred to a submerged recording chamber and continuously perfused with aCSF (flow rate: 1.5 ml/min). The bath temperature was maintained at 30-32° C. with an in-line solution heater and temperature controller (TC-344B, Warner Instruments). Identification of slice subfields and electrode positioning were done with 4× and 40× water immersion objectives on an upright microscope equipped with differential interference contrast and fluorescence optics under infrared illumination (BX5IWI, Olympus) and video observation (C3077-71 CCD camera, Hamamatsu Photonics). All recordings were made using Axopatch 200B amplifier (Molecular Devices). Data acquisition and stimulation protocols were performed with the Digidata 1440A Series interface and pClamp 10 software (Molecular Devices). Data were filtered at 1 kHz, digitized at 10 kHz, and analyzed both online and offline. Field recordings were made using glass pipettes filled with aCSF (tip resistance 2-5 MΩ) and placed in the stratum radiatum of the CA1 region. Field excitatory synaptic potentials (fEPSPs) were evoked by stimulation of the Schaffer collaterals with a bipolar tungsten electrode (FHC, USA) connected to a constant current isolated stimulator (Digitimer). The stimulation intensity that produced one-third of the maximal response (fEPSP slope), was used for the test pulses, Long Term Potentiation (LTP) induction and paired-pulse facilitation protocols. Data were analyzed by assessing the initial phase of the fEPSP slope. The initial linear slope of fEPSPs was used as a measure of the post-synaptic response and fiber volley (FV) amplitude as a measure of the strength of the pre-synaptic activation (i.e., axonal depolarization). In detail, synaptic function was evaluated by constructing input-output relationships in which the fEPSP slope measures were plotted against either stimulus intensity or fiber volley amplitude. Before the LTP induction protocol, to evaluate the basal synaptic transmission, I/O curves were obtained: i) by recording fEPSPs induced by presynaptic stimulation at intensities ranging from 20 to 300 gA (in increments of 30 or 50 gA; stimulus rate of 1 pulse every 20 s); ii) by plotting fEPSP slopes against the amplitudes of presynaptic fiber volley. On the same synapses, paired pulse facilitation (PPF) was assessed at inter-stimulus intervals ranging from 20 to 500 ms. For LTP recordings, stable baseline responses to test stimulations (1 pulse every 20 s for 10 min) were recorded and then high-frequency stimulation (HFS) was delivered (4 trains of 50 stimuli at 100 Hz, 500 ms each, repeated every 20 s, double-pulse width). Responses to test pulse were recorded every 20 s for 60 min to assess LTP. LTP magnitude was expressed as the percentage change in the mean fEPSP slope normalized to baseline values (i.e., mean values for the last 5 minutes of recording before HFS, taken as 100%).

Novel Object Recognition Test (NOR)

Two weeks after 12A12 mAb injection, mice run the novel object recognition (NOR) (Bevins et al., 2006) task to check the hippocampal-dependent episodic memory (Antunes et al., 2012; Akkerman et al., 2012). The entire task was performed in three consecutive sessions during the same day, according to previous protocol (Borreca et al., 2018). Mice were first transferred to the experimental room and left undisturbed in their home-cage for 30-min acclimation in the new environment. During the first habituation session, each mouse was placed for 10-min in the testing arena (empty cubic box 50×50×30 cm made of white opaque plastic material) and then returned to the home-cage for a 10-min interval. Then, each mouse was placed in the testing arena for sample trial, which consisted in the exposition of two identical objects (objects 1 and 2) for 10-min period. Objects were either two colored plastic cubes (5×5×5 cm) or two glass cylinders (8 cm high and 5 cm diameter) and were presented according to a random schedule. The objects were cleaned with 10% ethanol before the third session. Mouse's interest for the objects was measured as exploration, which was defined as time mice spent sniffing or touching the objects (Left and Right objects, LO and RO) with nose and/or forepaws. At the end of sample trial, mice were back in their home-cage and were left undisturbed for 60-min inter trial interval. During the following test trial, each mouse was back in the testing arena where one of the two objects remained unvaried (LO, familiar object FO) while the other one (RO) was replaced with a different one (novel object NO). In this session, objects exploration was measured as above and the interest for the NO was inferred by calculating the preference index (NO/FO+NO ratio). A preference index above 50% indicates that the NO was preferred to FO, while preference index of 50% indicates that mice spent the same amount of time in exploration of the two objects. The mice were allowed to explore the apparatus for a total of five minutes while being recorded by an overhead camera positioned above the testing arena, and then removed from the apparatus. An experimenter blind to experimental conditions manually assessed mice exploratory behavior toward the objects. General exploratory and locomotory activities were assessed through Noldus Ethovision system (The Netherlands).

Object Place Recognition Test (OPR)

The object place recognition (OPR) paradigm were carried out as following: a common habituation phase, a training phase and a test phase. This behavioural task involves the activity of the hippocampus and is used to test short-term memory. The animals, which underwent the NOR paradigm with a training and test session, were tested in the OPR paradigm twenty-four hours later, with a separated training and test session. The objects used for the OPR were different from those used previously for the NOR test. During the habituation phase, animals were placed for five days, 10 minutes per day, into a square-shaped grey arena (44×44 cm). In the training phase, animals were exposed to two identical objects for 10 minutes. In the training phase, lasting 10 minutes for each animal, two identical objects were placed nearby the corners of arena. Objects (Left and Right objects, LO and RO) were either two colored plastic cubes (5×5×5 cm) or two glass cylinders (8 cm high and 5 cm diameter) and were presented according to a random schedule. At the end of sample trial, mice were back in their home-cage and were left undisturbed for 60-min inter trial interval. During the following test trial, each mouse was back in the testing arena where one of the two objects (RO) remained unvaried (Stationary object, SO) while the other one (LO) was moved in a different position (Displaced object, DO). Mice were then allowed to explore the objects for 10 minutes. Mice's interest for the objects was measured as exploration, which was defined as time mice spent sniffing or touching the objects with nose and/or forepaws or pointing toward it at a distance <2 cm. Time interacting with the objects was scored, and a preference index was calculated as the ratio between time exploring the new/displaced object and total exploration time, multiplied by 100. The floor was covered with wooden beddings (which were changed between each animal) and different cues were positioned on the internal walls of the arena in order to provide mice with spatial points of reference for the OPR. The objects were cleaned with 70% ethanol and water and dried between trials, in order to avoid possible confounding effects. For the training and test phase, mice were recorded with an infrared camera placed above the arena and the analysis was carried out with ANY-Maze™ (Stoelting).

Spontaneous Alternation (Y-Maze) Test

Evaluation of short-term working memory was carried out using the spontaneous alternation version of the Y-maze, which involves different brain structures ranging from the hippocampus to the prefrontal cortex. The apparatus consists of a black opaque Perspex plexiglass Y-shaped maze with 3 arms (A, B, and C) containing a visual cue (arm dimensions; 15 cm×10 cm×10 cm) and divided by 120° angles. Each animal was placed in turn in arm A of the Y-maze and allowed to explore for 8 minutes and the arm entries made by each animal were recorded. Arm entry was defined as having all 4 paws in the arm. The entrance sequence, correct triplets and number of entrances were scored. An index of spontaneous alternation was calculated as the ratio between number of correct triplets (e.g. ABC) and total entrances minus 2, multiplied by 100 (Hiramatsu et al., 1997; Wall and Messier, 2002).

Golgi Cox Staining and Dendritic Spine Analysis

Sixty minutes after the retention test, mice (3 or 5 for each experimental condition) were treated with lethal dose of anesthetic (Zoletil/Rompun 800 mg/kg and 100 mg/Kg, respectively) and perfused transcardially with 0.9% saline solution (n=2 mice per group). Brains were dissected and immediately immersed in a Golgi-Cox solution (1% potassium dichromate, 1% mercuric chloride, and 0.8% potassium chromate) at room temperature for 6 days. On the seventh day, brains were transferred in a 30% sucrose solution for cryoprotection and then sectioned with a vibratome. Coronal sections (100 μm) were collected and stained according to (Borreca et al., 2018). Sections were stained through consecutive steps in water (1 minute), ammonium hydroxide (30 minutes), water (1 minute), developer solution (Kodak fix 100%, 30 minutes), and water (1 minute). Sections were then dehydrated through successive steps in alcohol at rising concentrations (50%, 75%, 95%, and 100%) before being closed with coverslip slide. For quantification of dendritic spines, images of pyramidal neurons from the CA1 region of the hippocampus were captured by selecting well-stained neurons randomly at 40× magnification with water immersion and for the analysis of dendritic spine density images were acquired randomly at 100× magnification with oil immersion. At least 5 neurons within each hemisphere were taken from each animal. On each neuron, five 30-100 μm dendritic segments of secondary and tertiary branch order of CA1 dendrites were randomly selecte. Spine density was measured online using the software Neurolucida (Microbrightfield) connected to an optical microscope DMLB Leica. counted using Neurolucida software. The criteria used for analyzing neurons are as follows: the pyramidal neurons had to be fully impregnated and located in the CA1 region of the hippocampus without truncated branches and the soma located centrally within the 120 μm section depth. The criteria for spines included impregnation intensity allowing visibility of spines, a low level of background, spines counted only on dendrites starting at more than 85 μm distal to the soma and after the first branch point. Only protrusions with a clear connection of the head of the spine to the shaft of the dendrite were counted as spines. Statistical comparisons were made on single neuron values obtained by averaging the number of spines counted on segments of the same neuron. Analysis was performed blindly, with the analyzer unaware of the experimental conditions. Spine density was calculated by quantifying the number of spines per measured length of dendrite and expressed as the number of spines per μm length of dendrite. The length of each dendritic segment used for spine densitometry was at least 20 μm but not greater than 50 μm in length.

Synaptosomes Preparation

Mouse hippocampal purified synaptosomes were prepared by homogenizing tissue in 10 volumes of 0.32 M sucrose, buffered to pH 7.4 with Tris-(hydroxymethyl)-amino methane [Tris, final concentration (f.c.) 0.01 M]. The homogenate was centrifuged at 1,000 g for 5 min and the supernatant was stratified on a discontinuous Percoll gradient (2%, 6%, 10% and 20% v/v in Tris-buffered sucrose) and centrifuged at 33.500 g for 5 min. The layer between 10% and 20% Percoll (synaptosomal fraction) was collected and washed by centrifugation. The synaptosomal pellets were resuspended in a physiological solution with the following composition (mM): NaCl, 140; KCl, 3; MgSO4, 1.2; CaCl₂), 1.2; NaH2PO4, 1.2; NaHCO₃, 5; HEPES, 10; glucose, 10; pH 7.2-7.4. To ascertain whether fractionated preparations were really enriched in synaptic terminals and free of any contaminations from neuronal perikarya, Western blotting analysis was carried out to check the purity of samples by probing with antibodies against the presynaptic protein synaptophysin and cytosolic GAPDH, as previously reported (Corsetti et al., 2015).

Tissue Harvesting and Total Protein Lysates Preparation

Tissue sampling and total protein lysates preparation was carried out according to Castillo-Carranza et al., 2015 with some modifications. Briefly, animals were sacrificed by cervical dislocation to eliminate anesthesia-mediated tau phosphorylation (Panel et al., 2007), brains were collected and hippocampus were dissected and stored at −80° C. until use. For biochemical analysis, frozen hippocampi were diced and homogenized in phosphate buffered saline with a protease inhibitor mixture (Roche) and 0.02% NaN3 using a 1:3 (w/v) dilution. Samples were then centrifuged at 10,000 rpm for 10 min at 4° C. and the supernatants were collected.

Western Blot Analysis and Densitometry

Equal amounts of protein were subjected to SDS-PAGE 7.5-15% linear gradient or Bis-Tris gel 4-12% (NuPage, Invitrogen). After electroblotting onto a nitrocellulose membrane (Hybond-C Amersham Biosciences, Piscataway, N.J.) the filters were blocked in TBS containing 10% non-fat dried milk for 1 h at room temperature or overnight at 4° C. Proteins were visualized using appropriate primary antibodies. All primary antibodies were diluted in TBS and incubated with the nitrocellulose blot overnight at 4° C. Incubation with secondary peroxidase coupled anti-mouse, anti-rabbit or anti-goat antibodies was performed by using the ECL system (Amersham, Arlington Heights, Ill., U.S.A.) In a few experiments, multiple normalizations of the same filter on different loading controls, such as β-III tubulin and GAPDH (glyceraldehyde 3-phosphate dehydrogenase), were carried. Final figures were assembled by using Adobe Photoshop 6 and Adobe Illustrator 10 and quantitative analysis of acquired images was performed by using ImageJ (http://imagej.nih.gov/ij/). The following antibodies were used: anti-Abeta/APP protein 6E10 (aa 4-9) mouse MAB1560 Chemicon (1:500); anti-pan tau protein H150 (aa 1-150 of N-terminus) rabbit sc-5587 Santa Cruz Biotechnology (1:1000); anti-pan tau protein (microtubule binding repeat) mouse DC25 T8201 Sigma Aldrich; neuronal marker beta III tubulin antibody mouse ab78078(clone 2G10) Abcam; GAPDH antibody (6C5) mouse sc-32233 Santa Cruz Biotechnology; Arc (activity-regulated cytoskeleton-associated protein) (C-7) mouse sc-17839 Santa Cruz Biotechnology; Glial Fibrillary Acidic Protein (GFAP) antibody rabbit Z0334 Dako; Iba1 antibody rabbit Wako 016-20001 (for WB) and 019-19741 (for IF); neuronal marker β III tubulin antibody mouse Abeam (clone 2G10) ab78078.

Statistical Analysis

Data were expressed as means±S.E.M. and were representative of at least three separate experiments (n=independent experiments), including at least 3 or 4 animals for each experimental condition. Statistically significant differences were calculated by one-way or two-way analysis of variance (ANOVA) followed by Bonferroni and Fisher post-hoc tests, respectively, for multiple comparison among more than two groups. p<0.05 was accepted as statistically significant (*p<0.05; **p<0.01; ***p<0.0005; *** p<0.0001). All statistical analyses were performed using GraphPad Prism 7 software.

### EXAMPLES

### Example 1. Intravenously Injected Anti-NH₂Htau 12A12mAb is Detected and Biologically-Active (Target-Engagement/Binding) In Vivo, in the Hippocampus of Immunized Mice

Tg2576 and 3XTg mice—two well-established animal AD models (Hsiao et al., 1996; Oddo et al., 2003) which express the human APP695 with Swedish mutations (K670N-M671L), alone or in combination with MAPT P301Land PSEN1 M146V respectively—were analyzed because these transgenic animals are recognized to display tau-dependent, hippocampus-based cognitive impairments (Castillo-Carranza et al., 2015; Oddo et al., 2006; Amar et al., 2017). The hippocampal parenchyma was examined in the present study, since this vulnerable cerebral area: (i) selectively and disproportionally degenerates at early stages of MCI (Mild Cognitive Impairment) prior to the clinical diagnosis of full-blown dementia (West et al., 1994, Honer et al., 1992; Gomez-Isla et al., 1996; Kordower et al., 2000; Scheff et al., 2006a-b); (ii) preferentially develops tau neuropathology which critically subserves the transition from normal aging to MCI (Braak and Braak, 1991, Arrigada et al., 1992 Markesbery et al., 2006, 2010; Guillozet et al., 2003). To address the possible benefits offered by the immunological targeting of the NH₂htau on early neuropathological determinants and cognitive deficits, inventors infused 6-month-old transgenic AD animal models from these two different strains (Tg-AD) over 14 days with two weekly injections of 12A12 mAb (30 μg/dose) which were administered on two alternate days to the lateral vein of the tail. Both littermate age-matched wild-type and naive (i.e. nonimmunized) transgenic AD counterparts—which were infused under the same experimental conditions with vehicle (saline) only—were also included. Because the i.v.-infused 12A12 mAb has to reach the brain in order to neutralize the pathological NH₂htau accumulating in both AD transgenic models, in order to test whether it gained access to murine cerebral parenchyma under our immunization regimen, inventors first carried out Western blotting analysis by probing with anti-mouse IgG as primary antibody on hippocampal protein extracts from the three experimental groups (wild-type, naive Tg-AD, Tg-AD+mAb). As shown in FIG. 1A, 3XTg animals which were systemically injected for 14 days with 12A12mAb, exhibited high levels of cerebral mouse IgG when compared to not-vaccinated controls. This finding is in line with previous reports on the ability of other, intravenously-administered anti-tau antibodies to cross the blood brain barrier (BBB) of diseased mice, likely owing to its age-related impairment and increased permeability (Asuni et al., 2007; Mably et al., 2015). No specific immunoreactivity was found in age-matched wild type and naive 3XTg groups which were infused with saline alone under the same experimental conditions. Next, to confirm that peripherally-delivered 12A12mAb was actually able to bind the NH₂htau in vivo, inventors carried out Enzyme-Linked Immunosorbent Assay (ELISA) test on TBS-soluble fractions isolated from hippocampi of wild type, naive 3XTg, 3XTg+mAb animals after 14 days i.v. injection. Healthy, littermate wild-type mice infused with 12A12mAb under the same experimental conditions (wild-type+mAb) were also included to ascertain whether 12A12mAb could enter the brain from periphery despite the intact BBB and/or the lack of tau pathology within the CNS. It's worth underlying that: (i) the ELISA test aimed at assessing the cerebral amount of injected 12A12 mAb is based on the plate-immobilized synthetic NH₂26-44 which, being the minimal AD-relevant (Borreca et al., 2018) active moiety of the parental longer NH₂ 26-230 (Amadoro et al., 2004, 2006), was used as catching peptide; (ii) only the free (i.e. unoccupied) antibody can readily bind to its immobilized specific antigen and be measured, whereas the tau-bound antibody is not detectable. As shown in FIG. 1B, a sizeable proportion of the injected 12A12 mAb was unbound and active (antigen-binding competent) in 3XTg brains, being able to recognize the synthetic plate-immobilized recombinant NH₂26-230 antigenic peptide. Interestingly, the levels of i.v.-administered 12A12mAb were significantly lower in 3XTg+mAb experimental group (two-way ANOVA analysis followed by Bonferroni post-hoc test for multiple comparisons, genotype X treatment interaction F=(1,24)=28.92 p<0.0001; wild-type+saline versus Tg-AD+saline n.s. p>0.99; *****p<0.0001 for all other pair comparisons) than in wild-type+mAb littermates treated under the same experimental conditions, indicating that higher fraction of this antibody is actually bound in vivo to the endogenously-generated NH₂htau antigen, and then less available for capture in ELISA assay, into the hippocampi from diseased 3XTg than in healthy wild-type controls. Collectively, these findings demonstrated that 12A12mAb: (i) was actively up-taken into the brain after i.v. injection because an appreciable percentage of free and antigen binding-competent antibody was present into hippocampus both from healthy wild-type and 3XTg immunized mice, regardless the integrity of their BBB and/or the presence of tau pathology; (ii) did not nonspecifically interact, neither in wild-type nor in 3XTg, with the large amount of intracellular full-length normal tau which is routinely released during procedure of samples homogenization, in line with our previous in vivo observations advocating its cleavage-specificity towards the NH₂htau truncated fragment (Amadoro et al., 2012); (iii) was not limiting because it is detectable and active in mice brains after immunization regimen. Similar results were also found in Tg2576 animals from the other genetic background which were analyzed and treated under the same experimental conditions.

### Example 2. 12A12 mAb Passive Vaccination Reduces Both the Pathological Tau and Soluble, Prefibrillar as Species into Synaptic Compartments from Treated AD Transgenic Mice at Prodromal Stage of Neuropathology

Co-occurrence between tau and Aβ pathology has been described to take place within neuronal processes and nerve ending compartments at early stages of the AD development (Takahashi et al., 2010; Amadoro et al., 2012; Spires-Jones and Hyman, 2014; Rajmohan and Reddy, 2017). In preclinical models of Tg2576 and 3xTg, Aβ exerts its synaptotoxicity, at least in part, via tau, but both separate and synergistic neurodegenerative mechanisms have been also described in these two experimental paradigms (Nisbet and Gotz, 2015). Therefore, having established that hippocampus is successfully targeted in vivo following i.v. 12A12mAb mAb infusion, inventors examined the effect of immunization regimen on the APP amyloidogenic processing-derivatives, the full-length total tau and its AD-relevant abnormally-hyperphosphorylated species. To this aim, Western blotting SDS-PAGE analyses were carried on hippocampal synaptosomal preparations from the three experimental groups (wild-type, naive Tg-AD, Tg-AD+mAb) of both genetic background (3-month-old pre-symptomatic Tg2576 and 3XTg, respectively) by probing with specific commercial antibodies detecting the site-specific tau hyperphopshorylation at Ser202/Thr205 epitope (AT8) and the accumulation of soluble 6E10-positive Ab monomeric and oligomeric species, which are two neurochemical hallmarks known to accumulate and affect the AD nerve terminals (Braak and Del Tredici, 2015). First of all and consistent with previous investigations from rodent preparations (Rohn et al., 2002; Corsetti et al., 2008) and human nerve terminals specimens (Amadoro et al., 201, 2012; Corsetti et al., 2015; Sokolow et al., 2015), the steady-state expression level of the neurotoxic 20-22 kDa NH₂htau truncated fragment significantly increased under diseased conditions (FIG. 2-3), as shown following quantitative analysis of synaptic-enriched fractions from saline-treated, naive Tg-AD mice in comparison to nontransgenic littermate wild-type controls (one-way analysis of variance (ANOVA) followed by Bonferroni post-hoc test for multiple comparisons F(2,18)=135.8 p<0.0001 Tg2576; F(2,18)=72.84 p<0.0001 3XTg; ****p<0.0001 for all pair comparisons from Tg2576; ****p<0.0001 3xTg versus wild-type). Interestingly, the passive vaccination with 12A12 mAb was able to significantly reduce the synaptic load of 20-22 kDa NH₂htau form(s) in Tg-AD animals from both strains, by successfully engaging/intercepting its target into hippocampus with consequent neutralization/clearance in vivo (one-way ANOVA followed by Bonferroni post-hoc test ****p<0.0001 for all pair comparisons from Tg2576; ***p=0.0005 3xTg+mAb versus wild-type; ****p<0.0001 3xTg+mAb versus 3xTg). The AD-like pathognomonic hyperphosphorylation on residues Ser202 and Thr 2⁰⁷ detected by AT8 antibody—which binds both mouse and human proteins (Goedert et al., 1995)—was also strongly inhibited in Tg-AD animals following 12A12mAb immunization (one-way ANOVA followed by Bonferroni post-hoc test F(2,21)=33.12 p<0.0001 Tg2576; F(2,18)=76.04 p<0.0001 3xTg; ****p<0.0001 Tg2576 versus wild-type; n.s. p=0.4689 Tg2576+mAb versus wild-type; ****p<0.0001 Tg2565+mAb versus Tg2576; ****p<0.0001 3xTg versus wild-type; n.s. p=0.1332 3xTg+mAb versus wild-type; ****p<0.0001 3xTg+mAb versus 3xTg), indicating that the anti-truncated tau antibody successfully downregulated the extent of tau neuropathology in vivo. Furthermore and in line with previous literature findings highlighting a mechanistic direct and/or indirect interaction between Aβ and tau pathology in vivo (Castillo-Carranza et al., 2015; Dai et al., 2017, 2018), i.v. administration of 12A12 mAb led to a dramatic decline and/or disappearance of the immunoreactivity levels of 6E10-positive harmful human-specific Aβ specie(s) in Tg-AD hippocampal synapses from treated experimental groups of both genetic backgrounds (i.e. monomer and low-molecular weight oligomers, Teich et al., 2015) (one-way ANOVA followed by Bonferroni post-hoc testF(2,18)=138.5 p<0.0001 Tg2576; F(2,18)=124.3 p<0.0001 3xTg; ****p<0.0001 Tg2576 versus wild-type; *p=0.0232 Tg2576+mAb versus wild-type; ****p<0.0001 Tg2576+mAb versus Tg2576; ****p<0.0001 for all pair comparisons from 3xTg). Notably, the steady-state expression level of total tau detected by H150 and DC25, two commercial anti-pan tau antibodies binding all both murine and human tau isoforms (Um et al., 2011, Lee et al., 2010; Zilka et al., 2006), was unchanged in synapses from AD transgenic animals after 12A12 mAb immunization regimen, with significantly higher level of total tau detected in 3xTg-AD genetic background in comparison wild-type littermates due to the presence of both endogenous and human transgenic protein (one-way ANOVA followed by Bonferroni post-hoc test F(2,18)=0.4618 p=0.6374 Tg2576; F(2,18)=32.03 p<0.0001 3xTg; n.s. p>0.999 for all pair comparisons from Tg2576; ***p=0.0001 3xTg versus wild-type; ****p<0.0001 3xTg+mAb versus wild-type; n.s. p=0.0728 3xTg+mAb versus 3xTg). These findings are consistent with in tau cleavage-specificity of 12A12mAb which selectively binds in vivo the neurotoxic NH₂htau truncated specie(s) (Amadoro et al., 2012; Corsetti et al., 2015) without cross-reaction with the full-length form of protein. Collectively, these findings demonstrated that: (i) when i.v. administrated to pre-symptomatic (3-month-old) Tg2576 and 3XTg, two well-established AD animal models showing tau-dependent neuropathology (Castillo-Carranza et al., 2015; Oddo et al., 2006; Amar et al., 2017), the cleavage-specific 12A12 mAb is able to reach an appreciable concentration into the hippocampal parenchyma ending up in an effective target engagement/neutralization in vivo (i.e., binding/interception of the pathologic 20-22 kDa NH₂htau form(s)); (ii) the antibody-mediated removal of the 20-22 kDa NH2htau form(s) positively influences the detrimental alterations of both APP and tau metabolism (i.e. AT8 tau hyperphosphrylation and Ab species accumulation) commonly occurring at the earliest stage of AD onset/progression into nerve endings; (ii) the 12A12mAb-mediated immunodepletion of the toxic 20-22 kDa NH2htau form(s) take places in the absence of any significant change in the local stability/turnover of normal full-length tau protein which is endowed with important physiological functions into synaptic compartments (Pooler et al., 2014; Regan et al., 2017) and whose reduction, even if partial, is known to be extremely harmful for post-mitotic neurons in vivo (Biundo et al., 2018; Velazquez and Oddo, 2018).

### Example 3. Cognitive Performance is Significantly Improved in Symptomatic AD Transgenic Mice after Passive Immunization with 12A12 mAb

Having established that classical molecular determinants underlying the phenotypic AD manifestations are strongly reduced at early/pre symptomatic stages of neuropathology following i.v. administration of 12A12 mAb, cognitive functioning of old Tg-AD animals (6-month-old Tg2576 and 3XTg mice, respectively) was tested after the same vaccination regimen (two weekly injections of 30 μg mAb/dose to the lateral vein of the tail for 14 days) by means of comprehensive behavioral test battery. The novel object recognition task (NOR) is a paradigm which is considered an appropriate readout for measures of learning/memory impairment in transgenic and non-transgenic animal models of tauopathies, including AD (Polydoro et al. 2009; Lanté et al 2015). Relevantly, the NOR behavioural test: (i) involves brain areas such as transentorhinal/entorhinal/perirhinal cortices and hippocampus which are pathologically relevant in this field, being affected by neurofibrillary tau changes at early stages of disease (Braak and Braak 1991; Bengoetxea et al., 2015; Sankaranarayanan et al 2015; Lasagna-Reeves et al. 2011, 2012) (ii) is a non-aversive learning paradigm, avoiding the potential confounds of using differential rewards or punishments, able to test the hippocampal-dependent episodic memory (Antunes et al., 2012; Leger et al., 2013) which is the first type of memory affected in AD patients (Grayson, et al., 2015; deToledo-Morrell et al., 2007; Salmon et al., 2009; Reed et al., 1997; Zola et al., 2001). Owing to innate and spontaneous preference of mice towards novelty, any increase in exploration of the novel object (NO) during the test session is to be ascribed to animal's ability in discriminating it from the familiar one (FO) and this parameter was quantified as preference/recognition index (RI), which is calculated as the percentage of time spent exploring the new object over the total time spent exploring the two objects. In the recognition session, a preference index for the NO above 50% indicated that the NO was preferred, below 50% that the FO was preferred, and at 50% that no object was preferred (Hammond et al., 2004). Interestingly, inventors found out that AD mice from two genetic backgrounds (Tg2576 and 3xTg, respectively) receiving 12A12mAb showed a significant rescue in short-term memory deficits when tested in this hippocampal-dependent task, being able to distinguish NO from FO (Tg2576+mAb RI=58.6%; 3xTg+mAb RI=66.41%) just as wild-type, healthy nontransgenic mice (B6SJL RI=59.44%; C57 RI=68.0%, respectively) (FIG. 4, 5). On the other hand, saline-treated/naive AD mice from both strains (Tg2576 and 3xTg, respectively) showed a poor performance in short-term memory NOR task because they spent the same time in exploring the NO versus the FO (Tg2576 RI=48.51; 3xTg RI=50.48, respectively). Accordingly, a two-way ANOVA statistical analysis of behavioural data (treatment×object discrimination) indicated significant difference between the three experimental groups of both strains analyzed (F_{(1;32)}=6.60 p=0.01 for Tg2576; F_{(2;56)}=3.4 p=0.04 for 3xTg) with the novel object being preferred from AD transgenic animals infused with mAb (Fisher post-hoc test NO vs FO=Tg2576+mAb: *** p<0.005, 3xTg+mAb: *** p<0.005) which behaved in the same manner of wild-type, nontransgenic ones endowed with cognitive-skills (Fischer post-hoc test NO vs FO B6SJL: **p<0.01, C57: ***p<0.005). Conversely, not-immunized AD mice from both genetic backgrounds did not discriminate between NO and FO object and displayed memory/learning impairment without any preference for NO (Fischer test Tg2576: p=0.61; 3xTg: p=0.32). Furthermore no significant difference (treatment×object discrimination) was measured during training phase among the three experimental groups from both strains which explored both objects for the same length of time and without any particular preference toward a side of the cage (two-way ANOVA analysis F_{(2;32)}=0.087 p=0.916 for Tg2576 background; F_{(2;52)}=1.09 p=0.34 for 3xTg mice; Fisher post-hoc test LO vs RO=B6SJL: p=0.53, Tg2576: p=0.20, Tg2576+mAb: p=0.30; Fischer post-hoc test LO vs RO C57: p=0.72, 3xTg: p=0.91, 3xTg+mAb: p=0.11). In addition to recognition memory, the hippocampal formation is also devoted to store information about places in the organism's environment, their spatial relations, and the existence of specific objects in specific places (spatial memory) (O'Keefe and Conway, 1978; Broadbent and Clark, 2004 Manns and Eichenbaum, 2009). Therefore, to deeply investigate the beneficial effect of 12A12mAb on AD-related cognitive deficits, immunized and not-immunized animals from the three experimental groups run the Novel Object Place (NOP) task, another hippocampal-dependent paradigm which examines their memory/learning ability not in the objects recognition but in its spatial relationships by calculating the time spent in in discriminating the spatially displaced “old familiar” object relative to the stationary “old familiar” object, (Ciernia and Wood 2014; Antunes and Biala, 2012). Rodents displayed a clear preference for the object moved to a novel place (displaced object, DO) in comparison to the object that remained in the same (familiar) place (stationary object, SO), which confirmed their ability for remembering which spatial locations have or have not been engaged earlier (Warburton et al., 2013). Again, cognitive impairment of mice from the two genetic backgrounds (Tg2576 and 3xTg, respectively) is relieved following i.v. 12A12mAb infusion because immunized animals were able to distinguish DO from SO (Tg2576 RI=73.26%; 3xTg RI=69.07%), performing in spatial novelty memory task just as wild-type, healthy nontransgenic ones (B6SJL RI=79.71%; C57 RI=71.48%, respectively). On the other hand, saline-treated/naive AD mice showed no preference for the moved object as they spent nearly equivalent amounts of time exploring the DO and SO, which confirmed that these not-immunized AD animals from both strains have object location memory dysfunction (Tg2576 home-cage RI=48.29; 3xTg home-cage RI=52.53%; respectively). Consistently, a two-way ANOVA statistical analysis of behavioural data (treatment×object discrimination) indicated significant difference between the three experimental groups in both animal strains analyzed (F_{(2;20)}=9.68 p=0.001 for Tg2576; F_{(2;50)}=33.11 p=0.00000 for 3xTg) with the DO being preferred from AD mice immunized by 12A12 mAb infusion (Fisher post-hoc test DO vs SO=Tg2576+mAb: *** p<0.005, 3xTg+mAb: *** p<0.005) which behaved in the same manner of wild-type, nontransgenic ones (Fischer post-hoc test DO vs SO B6SJL: ***p<0.005, C57: ***p<0.005). In contrast, naive Tg2576 and 3xTg mice displayed no difference between DO and SO object with no preference for DO (Fischer test Tg2576: p=0.76; 3xTg: p=0.35). Besides, no significant difference (treatment×object discrimination) was measured during training phase among the three experimental groups from both strains which explored both objects for the same length of time and without any particular preference toward a side of the cage (two-way ANOVA analysis F_{(2;2)}=0.47 p=0.63 for Tg2576 background; F_{(2;52)}=0.79 p=0.46 for 3xTg mice; Fisher post-hoc test LO vs RO=B6SJL: p=0.58, Tg2576: p=0.76, Tg2576+mAb: p=0.47; Fischer post-hoc test LO vs RO C57: p=0.24, 3xTg: p=0.86, 3xTg+mAb: p=0.68). After assessing the object discrimination and spatial memory, inventors also tested animals from the three experimental groups in, the spontaneous alternation, by employing the Y-Maze, an hippocampal-dependent episodic-like behavioral test for measuring the willingness of rodents to explore new environments Animals are started from the base of the apparatus in the form of a T placed horizontally and allowed to freely explore all three arms. The number of arm entries and the number of triads are recorded in order to calculate the percentage of alternation (Deacon and Rawlins 2006; Borchelt and Savonenko 2008) which is based on the act that the rodent tends to choose the arm not visited before, reflecting memory of the first choice (Paul and Abel, 2009). Interestingly, in line with previous literature findings (Yassine and Mathis, 2013; Deacon et al., 2008; King and Arendash 2002), spontaneous alternation task did not reliably detect progressive cognitive impairment in Tg2576 mice at 6 months of age because no difference was found in their working-memory performance in comparison to cognitively-intact, littermate wild-type ones, both in spontaneous alternation and total entries into the arms (spontaneous alternation one way ANOVA F_{(2;12)}=0.15 p=0.86; Fisher Post hoc wild-type vs Tg2576 p=0.99; Tg2576 vs Tg2576+mAb p=0.68; Total Entries F_{(2;12)}=0.28 p=0.76; Fisher Post Hoc wild-type vs Tg2576 p=0.81; Tg2576 vs Tg2576+mAb p=0.72). On the other hand, although cognitive impairment was clearly discernible in naïve AD experimental group from 3xTG genetic background when tested in Y maze task in comparison with age-matched wild-type one (Spontaneous alternation one way ANOVA F_{(2,28)}=7.44 p=0.025; Total entries F_{(2,28)}=18.01 p=0.00001), no significant improvement in working-memory/learning abilities was detected in 12A12mAb-injected, cognitively-impaired AD animals (Fischer Post Hoc Analysis Spontaneous alternation WT vs 3xTg p=0.03; WT vs 3xTg+mAb p=0.0007; 3xTg vs 3xTg+mAb p=0.17; Total entries WT vs 3xTg p=0.000005; WT vs 3xTg+mAb p=0.00015; 3xTg vs 3xTg+mAb p=0.19). In this framework, it's worth stressing that the transgenic Tg2576 mice expressing human mutant APP (K670N/M671L), in contrast to Tg3X harboring PS1(M146V), APP(Swe), and tau(P301L) transgenes, display an endogenous genetic background of murine not-mutated tau. Therefore, the discrepancy in results between two different genetic backgrounds, each having its own characteristics, may be due both to the more aggressive phenotype of the human tau-overexpressing 3xTg strain, which would require a more optimized immunization regimen to fully prevent and/or delay the its cognition symptomatology, and to the .complex and multifactorial nature underlying the AD pathology involving a wide range of strain-specific inflammatory, oxidative, neurodegenerative causative mechanisms. Importantly, no difference in cognitive performance were found when sham-immunized Tg-AD mice (i.e. animals injected with IgG control used at the same dosage) from both the genetic background were tested in behavioral tasks in comparison with their naïve transgenic counterpart. Active behavior, such as exploring a novel environment, induces the expression of the immediate-early gene Arc (activity-regulated cytoskeletal associated protein, or Arc/Arg3.1) in several brain regions, including the hippocampus. Arc messenger ribonucleicacid (mRNA) is quickly induced and dynamically up-regulated by behavioral experience and protein is translated into activated dendrites, being required for the memory consolidation of an early initial potentiation of synaptic transmission into a lasting form of long-term potentiation (LTP) (Path et al., 2006; Korb et al., 2011 Ramirez-Amaya et al., 2005, 2013). Interestingly and consistent with results from behavioural assessments (FIG. 4-5), by Western blotting analyses carried out on hippocampal synaptosomal-enriched preparations isolated from post-trained animals of the three experimental groups (FIG. 6), inventors found out that that the stimulus-driven, steady-state expression level of Arc is significantly upregulated in 12A12 mAb-immunized Tg2576 and 3XTg when compared to their saline-treated cognitively-impaired counterparts (one-way ANOVA followed by Bonferroni post-hoc test F(2,18)=215.7 p<0.0001 Tg2576; F(2,18)=95.45 p<0.0001 3XTg; ****p<0.0001 for all pair comparisons from both strains). Conversely and in line with their poor performance when tested in behavioural tasks (FIG. 4-5), naïve AD transgenic animals—which were not systemically infused with 12A12 mAb—displayed marked defects in the experience-dependent induction of Arc expression, and then in memory/learning consolidation, because the immunoreactivity signal of protein detectable in their synaptic fractions was significantly lower than that from healthy nontransgenic littermate wild-type controls (one-way ANOVA followed by Bonferroni post-hoc test ****p<0.0001 for all pair comparisons from both strains).

Collectively our results indicate that passive immunization with mAb12A12, which selectively targets the neurotoxic NH₂htau fragment(s), significantly improves cognitive performance in symptomatic (6-month-old) aged animals, by rescuing their instinctual and innate preference for novelty (object recognition and object location skills) when tested in two different hippocampal-dependent behavioural tasks.

Then, it is concluded that the present findings are particularly relevant for tau physiopathology in the field of AD and other tauopathies, helping to design more beneficial tau-directed and disease-modifying in vivo curative approaches for these devastating neurological disorders.

Loss in Dendritic Spine Density is Prevented in the CA1 Region of Hippocampus from 12A12-Immunized 6-Month-Old Tg-AD Animals from Both Genetic Backgrounds

Dendritic spines, the sites of excitatory synapses, are cellular morphological specializations devoted to memory-forming processes in neurons (Segal, 2005). Being extremely dynamic structures, modification in their number or shape is an important index of synaptic plasticity occurring in response to external environmental inputs (Pignataro et al., 2015). As a consequence, loss of dendritic arborization (length/complexity) in vulnerable neuronal networks, although occurring along different spatio-temporal patterns in transgenic animal models, undoubtedly contributes to the progressing appearance of cognitive dysfunction in AD and other related dementias (Spires-Jones and Knafo, 2012; Knobloch and Mansuy, 2018). Therefore, in order to complement our behavioral findings, inventors assessed the neuroanatomical effect of passive immunization with 12A12mAb on dendritic connectivity from 6-month-old aged AD animals of the three experimental groups from both strains analyzed. To this aim, hippocampal sections were stained by Golgi-Cox impregnation procedure and quantitative assessment of dendritic spine density (number of spines per unit length) was performed along both apical and basal compartments of individual CA1 pyramidal neurons. As shown in FIG. 6 and in line with previous works reporting in Tg2576 AD mice an early decline in dendritic boutons which undergo dystrophy and shrinkage (Lanz et al., 2003; Jacobsen et al., 2006), the spine loss was detectable in apical compartments of CA1 hippocampal neurons at the age of 6-months when animals from this genetic background were compared to littermate nontransgenic wild-type. Importantly, the apical spine density was significantly ameliorated in 12A12mAb-immunized Tg2576 animals up to of the level of saline-injected cognitively-intact wild-type littermates (one-way ANOVA statistical analysis followed by Fisher Post hoc test (F_{(2, 31)}=6.4784, p=0.00446 Tg-AD+mAb versus wild-type p=0.4718; **p<0.01 wild-type versus Tg-AD p=0.007950; **p<0.01 Tg-AD+mAb versus Tg-AD p=0.001632), indicating treatment was effective in blocking the dendritic degeneration. Interestingly, no differences was detected when spines were counted in the basal compartment of CA1 neurons from the three experimental groups analyzed (one-way ANOVA Fisher Post hoc test F_{(2, 31)}=1.6505, p=0.20838; Tg-AD+mAb versus wild-type 0.50433, wild-type versus Tg-AD 0.07979; Tg-AD+mAb versus Tg-AD=0.25174), suggesting that age-related spine changes in Tg2576 mice initially involves the apical dendritic arbors with no apparent effect on basal dendrites of CA1 pyramidal neurons (which are more likely to be affected only later when their structural plasticity and stability (formation and elimination) is completely impaired due to the appearance of extensive plaque deposition (Spires-Jones et al., 2007). On the other hand, in stark contrast with previous literature findings (Bitner et al., 2010), inventors found out that the reduction in the spines density, both in apical and basal compartment of CA1 individual pyramidal neurons, already started from the age of 6 months in cognitively-impaired 3xTg AD mice (one-way ANOVA statistical analysis followed by Fisher Post hoc test apical: F_{(2, 21)}=5.8845, p=0.00935; basal: F_{(2, 21)}=4.5387, p=0.0230) which exhibited lower values in dendritic protrusions counts when compared with age-matched, nontransgenic wild-type. Remarkably, degeneration of dendritic spine structures was strongly increased in immunized Tg-AD mice, both in apical and basal compartment (apical: **p<0.01 Tg-AD+mAb versus Tg-AD 0.002714; *p<0.05 wild-type versus Tg-AD 0.04695; wild-type versus Tg-AD+mAb 0.2122; basal: **p<0.01 Tg-AD+mAb versus Tg-AD 0.008275; *p<0.05 wild-type versus Tg-AD 0.046432 wild-type versus Tg-AD+mAb 0.4333) indicating that 12A12Ab treatment—as result of increased afferent inputs to the CA1 from other neighboring hippocampal areas or as a local positive effect in the CA1 region—was able to mitigate the age-related pathology in post-synaptic connections in symptomatic 6-month-old 3xTg mice.

In Correlation with its Behavioural and Neuroanatomical Beneficial Action, 12A12 Immunization Also Prevents the AD-Related Electrophysiological Impairments in Aged Tg-AD Animal Models.

In order to investigate whether 12A12mAb immunization, in addition to its positive effects on AD-related behavioural and neurochemical abnormalities, was also able to exert an efficacious modulation of electrophysiological correlate(s) underlying the hippocampal memory/learning processes, inventors first recorded basal synaptic transmission and the strength of pre-synaptic Schaffer collaterals activation (i.e. axonal depolarization) from CA3-to-CA1 synapses in acute brain slides from 6-month-old wild-type and age-matched Tg2576 animals treated with saline-vehicle or 12A12mAb, respectively. To this aim, inventors first generated input/output (I/O) curves by stimulating the Schaffer afferents pathway every 20 s at increasing intensities and, then, by measuring the fiber volleys and field excitatory postsynaptic potentials (fEPSPs) elicited in the stratum radiatum of the CA1 area. As shown in FIG. 7 and in line with previous investigations reporting no change in basal synaptic transmission between 6-month-old Tg2576 and age-matched wild-type (Chapman et al., 1999; Nobili et al., 2017), plotting of fEPSP slopes against stimulus intensity and fEPSP slopes against fiber volley amplitude displayed a similar trend among the three experimental groups (Two-way ANOVA statistical analysis, stimulus intensity×experimental group, followed by Bonferroni post hoc test F(12,282)=0.8409 p=0.6082; n.s. p>0.6 for all comparisons), indicating no significant difference in input-output relationship. Before the LTP induction protocol, inventors also investigated the presynaptic function by assessing paired-pulse facilitation (PPF), a short-term plasticity paradigm which inversely depends on presynaptic changes in neurotransmitter release probability at nerve endings [164-169]. Again and consistent with previous results (Nobili et al., 2017; Jung et al., 2011) referring no significant dissimilarity in PPF between 6-month-old Tg2576 and littermate wild-type, short-term potentiation was almost identical among the three experimental groups (Two-way ANOVA statistical analysis, paired-pulse interval×experimental group, followed by Bonferroni post hoc test F(10,170)=0.51 p=0.8839; n.s. p>0.6 for all comparisons). In contrast, long-term potentiation (LTP)—which is a widely employed paradigm of synaptic plasticity occurring during learning/memory processes—is significantly compromised in 6-month-old Tg2576 mice in comparison to age-matched wild-type, suggesting that its disruption in this genetic AD animal model is more likely due to altered post-synaptic signaling pathways given that no alteration in PPF is contextually detected at this age (Nobili et al., 2017; Jacobsen et al., 2006; Jung et al., 2011; Jacobsen et al., 2006; Chapman et al., 1999). Interestingly, peripheral in vivo administration of 12A12mAb to Tg2576 animals was able to mitigate the hippocampal, disease-related LTP deficiency underlying their progressive memory and synaptic plasticity impairmentsand. In line with results from behavioural assessments, the LTP amplitude calculated after application of high-frequency stimulation (HFS) was significantly increased in 12A12mAb-immunized Tg-AD experimental group when compared to its naive congitively-impaired counterpart (one-way ANOVA statistical analysis followed by Bonferroni post hoc test F(2,21)=19.38 p<0.0001; ****p<0.0001 Tg2576 versus wild-type; *p<0.05 Tg2576+mAb versus Tg2576; Tg2576+mAb versus wild-type **p<0.01). Furthermore, in 6-month-old 3xTg mice (FIG. 8) and in contrast with results from Tg2576 showing that neither pre-synaptic activation (fiber volley amplitude) nor post-synaptic responses (fEPSP slope) were affected in this genetic background, the input-output relationship revealed a significant reduction of fEPSP slopes compared to littermate wild-type (Two-way ANOVA statistical analysis, stimulus intensity×experimental group, followed by Bonferroni post hoc test F(12,204)=5.812 p<0.0001, *p<0.05, **p<0.01 wild-type versus 3xTg). Interestingly, cumulative distributions of fEPSP slopes within the range of 100 μA and 300 μA of stimulus amplitude were shifted to higher values in 12A12mAb-immunized groups compared to their naive, cognitively-impaired counterpart, indicating that antibody treatment positively influenced the fast glutamatergic transmission in this genetic background (#p<0.05, ##p<0.01 3xTg versus 3xTg+mAb). Besides and consistent with data showing that the abnormalities in pre-synaptic release machinery are not detectable between 6-month-old 3xTg 6 and littermate wild-type (Oddo et al., 2003), no change in PPF short-term plasticity was found among the three experimental groups (two-way ANOVA statistical analysis, paired-pulse interval×genotype, followed by Bonferroni post hoc test F(12,198)=0.3464 p=0.9792 n.s. p>0.4 for all comparisons). On the other hand, 6-month-old 3xTg mice showed a lower post-tetanic potentiation compared to wild-type littermates, suggesting that, LTP reduction in this AD strain may be due to induction deficits (either pre and/or post-synaptic) owing to structural and functional modifications observed in their basal synaptic transmission and dendritic spine density. Finally and in a way similar to Tg2576, hippocampal slices from 6-month-old 12A12mAb-injected 3xTg mice displayed a strong potentiation after HFS bout, pointing to a strong protective action evoked in vivo by antibody treatment on the cellular/molecular correlate(s) of memory/learning processes (one-way ANOVA statistical analysis followed by Bonferroni post hoc testF(2,33)=7.018 p=0.0029; **p<0.01 3xTg versus wild-type; *p<0.05 3xTg+mAb versus 3xTg; 3xTg+mAb versus wild-type n.s. p>0.05). Taken together, results from electrophysiological recordings indicate that synaptic transmission disruption in hippocampal CA3-CA1 circuit from these two genetically distinct Tg-AD animal models, although appears to progress at different rate and involved non-overlapping causative mechanism(s), is significantly rescued following in vivo peripheral administration of 12A12mAb.

Expression Levels of Inflammatory Astroglial and Microglial Markers are Also Downregulated in 6-Month-Old 12A12-Immunized Tg-AD Animals Regardless the Genetic Background.

The inflammatory response which is one of the earliest manifestations of neurodegenerative tauopathies, including AD (Yoshiyama et al., 2007; Wes et al., 2014; Leyns et al., 2017). may act as a double-edged sword being either detrimental or protective depending on the context (Schlachetzki et al., 2009). On one hand, activated glial cells contribute to the AD pathogenesis by releasing inflammatory mediators such as inflammatory cytokines, complement components, chemokines, free radicals and gliotransmitters which in turn trigger neurodegenerative. On the other hand, astroglial reaction and microglia reaction is endowed with beneficial role by stimulating the digestion/clearance of pathological Ab and tau species accumulating into the typical disease-associated cerebral lesions, the senile plaques and neurofibrillary tangles. To get further insights into beneficial effect evoked by i.v. 12A12mAb-based immunization in Tg-AD mice, the extent of inflammatory response was checked on total extracts from hippocampi of the three experimental groups (wild-type, naive Tg-AD, Tg-AD+mAb) of both genetic background (6-month-old symptomatic Tg2576 and 3XTg, respectively). by Western blotting analysis with antibodies detecting the glial fibrillary acidic protein (GFAP) and Iba1, whose cell type-specific steady-state expression levels accepted to be indicative of active astrogliosis and microgliosis respectively (Sydow et al., 2016). As shown in FIG. 10, the immunoreactivity signals of both classical inflammatory markers were strongly increased in saline-treated, naive Tg-AD mice in comparison to nontransgenic littermate wild-type controls, in line with previous findings reporting a prominent astrocytic and microglial activation in hippocampal parenchyma from these transgenic animal models (Olabarria et al., 2010, 2011; Leyns et al., 2017). Remarkably, the gliosis detected in 12A12mAb-injected Tg-AD turned out to be significantly downregulated compared to their littermate naïve counterparts (one-way ANOVA statistical analysis followed by Bonferroni post hoc test GFAP: F(2,18)=15.32 p=0.0044; *p<0.05 3xTg versus wild-type; **p<0.01 3xTg+mAb versus 3xTg; n.s. p>0.05 3xTg+mAb versus wild-type; Iba1 F(2,18)=110.6 p<0.0001 ***p<0.0005 3xTg versus wild-type; ***p<0.0005 3xTg+mAb versus 3xTg; **p<0.01 3xTg+mAb versus wild-type; GFAP F(2,19)=231.3 p<0.0001 *p<0.05 Tg2576 versus wild-type; *p<0.05 Tg2576+mAb versus Tg2576; n.s. p>0.05 Tg2576+mAb versus wild-type; Iba1 F(2,19)=10.67 p=0.0106 *p<0.05 Tg2576 versus wild-type; *p<0.05 Tg2576+mAb versus Tg2576; n.s. p>0.05Tg2576+mAb versus wild-type), in line with the findings that antibody-mediated targeting of pathological tau in vivo does not necessarily required engagement of microglia that may induce deleterious neuroinflammation (Lee et al., 2016; ) and that the neuroprotective mechanism action evoked by tau-based immunotherapy is more likely to rely on the neutralization of toxic extracellular species and/or on preventing their uptake by neurons (Congdon et al., 2013; Gu et al., 2013). Taken together these findings indicate that i.v. delivery of 12A12mAb into hippocampus: (i) is avoid of potentially adverse inflammatory effects which are associated to classical immunization regimen and due of excessive microglial activation with increased phagocytotic activity considered to be one of mechanisms driving the antibody-mediated cerebral clearance of the pathological tau and Ab (Wilcock et al., 2004); (ii) globally limits the local activation of inflammatory response which is per se is both a consequence to the disease process and a contributor to the synaptic pathology and neuronal damage (Perry et al., 2010; Zotova et al., 2010 Schwab et al., 2010; Block et al., 2007; Edison et al., 2008; Yoshiyama et al., 2007).

### SELECTED BIBLIOGRAPHIC REFERENCES

Scheff S W, Price D A. Alzheimer's disease-related alterations in synaptic density: neocortex and hippocampus. J Alzheimers Dis. 2006; 9(3 Suppl):101-15. Review.
Scheff S W, Price D A, Schmitt F A, Mufson E J. Hippocampal synaptic loss in early Alzheimer's disease and mild cognitive impairment. Neurobiol Aging. 2006 October; 27(10):1372-84. Epub 2005 Nov. 9.
West M J, Coleman P D, Flood D G, Troncoso J C. Differences in the pattern of hippocampal neuronal loss in normal ageing and Alzheimer's disease. Lancet. 1994 Sep. 17; 344(8925):769-72.
J. H. Kordower, Y. Chu, G. T. Stebbins, et al. Ann Neurol, 49 (2000), pp. 202-213 Loss and atrophy of layer II entorhinal cortex neurons in elderly people with mild cognitive impairment
T. Gomez-Isla, J. L. Price, D. W. McKeel, J. C. Morris, J. H. Growdon, B. T. HymanProfound loss of layer II entorhinal cortex neurons occurs in very mild Alzheimer's disease J Neurosci, 16 (1996), pp. 4491-4500
Oddo S, Caccamo A, Shepherd J D, Murphy M P, Golde T E, Kayed R, Metherate R, Mattson M P, Akbari Y, LaFerla F M. Triple-transgenic model of Alzheimer's disease with plaques and tangles: intracellular Abeta and synaptic dysfunction. Neuron. 2003 Jul. 31; 39(3):409-21.
Markesbery W R, Schmitt F A, Kryscio R J et al (2006) Neuropathologic substrate of mild cognitive impairment. Arch Neurol 63:38-46
Nelson P T, Braak H, Markesbery W R (2009) Neuropathology and cognitive impairment in Alzheimer disease: a complex but coherent relationship. J Neuropathol Exp Neurol 68:1-14
Guillozet A L, Weintraub S, Mash D C, Mesulam M M (2003) Neurofibrillary tangles, amyloid, and memory in aging and mild cognitive impairment. Arch Neurol 60:729-736
Braak H, Braak E (1991) Neuropathological stageing of Alzheimer-related changes. Acta Neuropathol 82:239-259
Musiek E S1, Holtzman D M1. Three dimensions of the amyloid hypothesis: time, space and ‘wingmen’. Nat Neurosci. 2015 June; 18(6):800-6. doi: 10.1038/nn.4018.
Zhou L, Mcinnes J, Wierda K, Holt M, Herrmann A G, Jackson R J, et al. Tau association with synaptic vesicles causes presynaptic dysfunction. Nat Commun 2017; 8: 15295. doi: 10.1038/ncomms15295.
Fomer S1, Baglietto-Vargas D1, Martini A C1, Trujillo-Estrada L1, LaFerla F M2. Synaptic Impairment in Alzheimer's Disease: A Dysregulated Symphony. Trends Neurosci. 2017 June; 40(6):347-357. doi: 10.1016/j.tins.2017.04.002. Epub 2017 May 8.
Bokde A L1, Ewers M, Hampel H. Assessing neuronal networks: understanding Alzheimer's disease. Prog Neurobiol. 2009 October; 89(2):125-33. doi: 10.1016/j.pneurobio.2009.06.004. Epub 2009 Jun. 26.
Mangialasche F1, Solomon A, Winblad B, Mecocci P, Kivipelto M. Alzheimer's disease: clinical trials and drug development. Lancet Neurol. 2010 July; 9(7):702-16. doi: 10.1016/S1474-4422(10)70119-8.
Chen Z, Mengel D, Keshavan A, Rissman R A, Billinton A, Perkinton M, et al. Learnings about the complexity of extracellular tau aid development of a blood-based screen for Alzheimer's disease. Alzheimers Dement 2018; pii: S1552-5260(18)33561-1. doi: 10.1016/j.jalz.2018.09.010.
Avila J1,2, Pallas N1,2, Bolós M1,2, Sayas C L3, Hernandez F1,2. Intracellular and extracellular microtubule associated protein tau as a therapeutic target in Alzheimer disease and other tauopathies. Expert Opin Ther Targets. 2016 June; 20(6):653-61. doi: 10.1517/14728222.2016.1131269. Epub 2016 Feb. 16.
Sebastián-Serrano Á1,2, de Diego-Garciá L3,4, Díaz-Hernández M5,6. The Neurotoxic Role of Extracellular Tau Protein. Int J Mol Sci. 2018 Mar. 27; 19(4). pii: E998. doi: 10.3390/ijms19040998.
Amadoro G, Corsetti V, Atlante A, Florenzano F, Capsoni S, Bussani R, et al. Interaction between NH2-tau fragment and Aβ in AD mitochondria contributes to the synaptic deterioration. Neurobiol Aging 2012; 33: 833.e1-25. doi: 10.1016/j.neurobiolaging.2011.08.001.
Bloom G S. Amyloid-β and tau: the trigger and bullet in Alzheimer disease pathogenesis. JAMA Neurol. 2014 April; 71(4):505-8. doi: 10.1001/jamaneurol.2013.5847. Review.
Crimins J L, Pooler A, Polydoro M, Luebke J I, Spires-Jones T L. The intersection of amyloid β and tau in glutamatergic synaptic dysfunction and collapse in Alzheimer's disease. Ageing Res Rev. 2013 June; 12(3):757-63. doi: 10.1016/j.arr.2013.03.002. Epub 2013 Mar. 22. Review.
King D L1, Arendash G W. Behavioral characterization of the Tg2576 transgenic model of Alzheimer's disease through 19 months. Physiol Behav. 2002 Apr. 15; 75(5):627-42.
Yassine N, Lazaris A, Dorner-Ciossek C, Despres O, Meyer L, Maitre M, et al. (2013) Detecting spatial memory deficits beyond blindness in tg2576 Alzheimer mice. Neurobiol Aging 34: 716-730. 10.1016/j.neurobiolaging.2012.06.016[PubMed] [CrossRef]
Fuyuki Kametani and Masato Hasegawa Reconsideration of Amyloid Hypothesis and Tau Hypothesis in Alzheimer's Disease Front Neurosci. 2018; 12: 25.
Deacon R M and Rawlins J N (2006) T-maze alternation in the rodent. Nat Protoc 1: 7-12. [PubMed]
Antunes M and Biala G (2012) The novel object recognition memory: neurobiology, test procedure, and its modifications. Cogn Process 13: 93-110. 10.1007/s10339-011-0430-z [PMC free article] [PubMed] [CrossRef]
Borchelt D R and Savonenko A V (2008) Chapter 5.5 Transgenic mouse models of Alzheimer's disease and episodic-like memory In: Ekrem Dere A. E. L. N. and Joseph P. H., editors. Handbook of Behavioral Neuroscience. Elsevier; pp. 553-573.
Deacon R M, Cholerton L L, Talbot K, Nair-Roberts R G, Sanderson D J, Romberg C, et al. (2008) Age-dependent and -independent behavioral deficits in Tg2576 mice. Behav Brain Res 189: 126-138. 10.1016/j.bbr.2007.12.024[PubMed] [CrossRef]
King D L, Arendash G W, Crawford F, Sterk T, Menendez J, Mullan M J (1999) Progressive and gender-dependent cognitive impairment in the APP(SW) transgenic mouse model for Alzheimer's disease. Behav Brain Res 103: 145-162. [PubMed]
Broadbent N J1, Squire L R, Clark R E. Spatial memory, recognition memory, and the hippocampus. Proc Natl Acad Sci USA. 2004 Oct. 5; 101(40):14515-20. Epub 2004 Sep. 27
Segal M1. Dendritic spines and long-term plasticity. Nat Rev Neurosci. 2005 April; 6(4):277-84.
Luebke J I, Weaver C M, Rocher A B, Rodriguez A, Crimins J L, Dickstein D L, Wearne S L, Hof P R. Dendritic vulnerability in neurodegenerative disease: insights from analyses of cortical pyramidal neurons in transgenic mouse models. Brain Struct Funct. 2010; 214:181-199. doi: 10.1007/s00429-010-0244-2. [PMC free article] [PubMed] [CrossRef]
Bittner T1, Fuhrmann M, Burgold S, Ochs S M, Hoffmann N, Mitteregger G, Kretzschmar H, LaFerla F M, Herms J. Multiple events lead to dendritic spine loss in triple transgenic Alzheimer's disease mice. PLoS One. 2010 Nov. 16; 5(11):e15477. doi: 10.1371/journal.pone.0015477.
Lanz T A, Carter D B, Merchant K M. Dendritic spine loss in the hippocampus of young PDAPP and Tg2576 mice and its prevention by the ApoE2 genotype. Neurobiol Dis. 2003; 13:246-253. [PubMed]
Jacobsen J S, Wu C C, Redwine J M, Comery T A, Arias R, et al. Early-onset behavioral and synaptic deficits in a mouse model of Alzheimer's disease. Proc Natl Acad Sci USA. 2006; 103:5161-5166. [PMC free article] [PubMed]
Spires-Jones T L1, Meyer-Luehmann M, Osetek J D, Jones P B, Stern E A, Bacskai B J, Hyman B T. Impaired spine stability underlies plaque-related spine loss in an Alzheimer's disease mouse model. Am J Pathol. 2007 October; 171(4):1304-11. Epub 2007 Aug. 23
Spires-Jones T1, Knafo S. Spines, plasticity, and cognition in Alzheimer's model mice. Neural Plast. 2012; 2012:319836. doi: 10.1155/2012/319836. Epub 2011 Nov. 28.
Nussbaum J M1, Schilling S, Cynis H, Silva A, Swanson E, Wangsanut T, Tayler K, Wiltgen B, Hatami A, Rönicke R, Reymann K, Hutter-Paier B, Alexandru A, Jagla W, Graubner S, Glabe C G, Demuth H U, Bloom G S. Prion-like behaviour and tau-dependent cytotoxicity of pyroglutamylated amyloid-β. Nature. 2012 May 2; 485(7400):651-5. doi: 10.1038/nature11060.
Regan P, Whitcomb D J, Cho K. Physiological and Pathophysiological Implications of Synaptic Tau. Neuroscientist. 2017 April; 23(2):137-151. doi: 10.1177/1073858416633439. Epub 2016 Jul. 7. Review.
Pooler A M, Noble W, Hanger D P. A role for tau at the synapse in Alzheimer's disease pathogenesis. Neuropharmacology. 2014 January; 76 Pt A:1-8. doi: 10.1016/j.neuropharm.2013.09.018. Epub 2013 Sep. 25. Review.
Rajmohan R, Reddy P H. Amyloid-Beta and Phosphorylated Tau Accumulations Cause Abnormalities at Synapses of Alzheimer's disease Neurons. J Alzheimers Dis. 2017; 57(4):975-999. doi: 10.3233/JAD-160612. Review.
Takahashi R H1, Capetillo-Zarate E, Lin M T, Milner T A, Gouras G K. Co-occurrence of Alzheimer's disease β-amyloid and τ pathologies at synapses. Neurobiol Aging. 2010 July; 31(7):1145-52. doi: 10.1016/j.neurobiolaging.2008.07.021. Epub 2008 Sep. 3.
Spires-Jones T L, Hyman B T. The intersection of amyloid beta and tau at synapses in Alzheimer's disease. Neuron. 2014 May 21; 82(4):756-71. doi: 10.1016/j.neuron.2014.05.004. Review.
Knobloch M1, Mansuy I M. Dendritic spine loss and synaptic alterations in Alzheimer's disease. Mol Neurobiol. 2008 February; 37(1):73-82. doi: 10.1007/s12035-008-8018-z. Epub 2008 Apr. 26.
Jung J. H., An K., Kwon O. B., Kim H. & Kim J.-H. Pathway-specific alteration of synaptic plasticity in Tg2576 mice. Mol. Cells 32, 197-201 (2011).
Jacobsen J S1, Wu C C, Redwine J M, Comery T A, Arias R, Bowlby M, Martone R, Morrison J H, Pangalos M N, Reinhart P H, Bloom F E. Early-onset behavioral and synaptic deficits in a mouse model of Alzheimer's disease. Proc Natl Acad Sci USA. 2006 Mar. 28; 103(13):5161-6. Epub 2006 Mar. 20.
Chapman P. F. et al. . Impaired synaptic plasticity and learning in aged amyloid precursor protein transgenic mice. Nat. Neurosci. 2, 271-276 (1999). [PubMed]
Andrew F. Teich, * Mitesh Patel, and Ottavio ArancioA Reliable Way to Detect Endogenous Murine β-Amyloid PLoS One. 2013; 8(2): e55647. Published online 2013 Feb. 1. doi: 10.1371/journal.pone.0055647
Um H S1, Kang E B, Koo J H, Kim H T, Jin-Lee, Kim E J, Yang C H, An G Y, Cho I H, Cho J Y. Treadmill exercise represses neuronal cell death in an aged transgenic mouse model of Alzheimer's disease. Neurosci Res. 2011 February; 69(2):161-73. doi: 10.1016/j.neures.2010.10.004. Epub 2010 Oct. 20.
Lee D C1, Rizer J, Selenica M L, Reid P, Kraft C, Johnson A, Blair L, Gordon M N, Dickey C A, Morgan D. LPS-induced inflammation exacerbates phospho-tau pathology in rTg4510 mice. J Neuroinflammation. 2010 Sep. 16; 7:56. doi: 10.1186/1742-2094-7-56.
Zilka N1, Filipcik P, Koson P, Fialova L, Skrabana R, Zilkova M, Rolkova G, Kontsekova E, Novak M. Truncated tau from sporadic Alzheimer's disease suffices to drive neurofibrillary degeneration in vivo. FEBS Lett. 2006 Jun. 26; 580(15):3582-8. Epub 2006 May 22.
Yoshiyama Y, Higuchi M, Zhang B, et al (2007) Synapse Loss and Microglial Activation Precede Tangles in a P301S Tauopathy Mouse Model. Neuron 53:337-351. doi: 10.1016/j.neuron.2007.01.010
Sydow A, Hochgräfe K, Könen S, et al (2016) Age-dependent neuroinflammation and cognitive decline in a novel Ala152Thr-Tau transgenic mouse model of PSP and AD. Acta Neuropathol Commun 4:17. doi: 10.1186/s40478-016-O281-z
Wes P D, Easton A, Corradi J, et al (2014) Tau overexpression impacts a neuroinflammation gene expression network perturbed in Alzheimer's disease. PLoS One 9: . doi: 10.1371/journal.pone.0106050
Leyns C E G, Holtzman D M (2017) Glial contributions to neurodegeneration in tauopathies. MolNeurodegener 12:50. doi: 10.1186/s13024-017-0192-x
Perry V H, Nicoll J A, Holmes C (2010) Microglia in neurodegenerative disease. Nat Rev Neurol 6:193-201
Zotova E, Nicoll J A, Kalaria R et al (2010) Inflammation in Alzheimer's disease: relevance to pathogenesis and therapy. Alzheimers Res Ther 2:1
Schwab C, Klegeris A, McGeer P L (2010) Inflammation in transgenic mouse models of neurodegenerative disorders. Biochim Biophys Acta 1802:889-902
Block M L, Zecca L, Hong J S et al (2007) Microglia-mediated neurotoxicity: uncovering the molecular mechanisms. Nat Rev Neurosci 8:57-69
Yoshiyama Y, Higuchi M, Zhang B et al (2007) Synapse loss and microglial activation precede tangles in a P301S tauopathy mouse model. Neuron 53:337-351
Edison P, Archer H A, Gerhard A et al (2008) Microglia, amyloid, and cognition in Alzheimer's disease: an [11C] (R)PK11195-PET and [11C]PIB-PET study. Neurobiol Dis 32:412-419
Schlachetzki J C, Hüll M (2009) Microglial activation in Alzheimer's disease. Curr Alzheimer Res 6:554-563
Wilcock D M, Rojiani A, Rosenthal A et al (2004) Passive amyloid immunotherapy clears amyloid and transiently activates microglia in a transgenic mouse model of amyloid deposition. J Neurosci 24:6144-6151
Planel E, Richter K E, Nolan C E, Finley J E, Liu L, Wen Y, Krishnamurthy P, Herman M, Wang L, Schachter J B. et al. Anesthesia leads to tau hyperphosphorylation through inhibition of phosphatase activity by hypothermia. J Neurosci. 2007; 27:3090-3097. doi: 10.1523/JNEUROSCI.4854-06.2007.
M. Olabarria, H. N. Noristani, A. Verkhratsky, J. J. RodriguezConcomitant astroglial atrophy and astrogliosis in a triple transgenic animal model of Alzheimer's disea Glia, 58 (2010), pp. 831-838
M. Olabarria, H. N. Noristani, A. Verkhratsky, J. J. RodriguezAge-dependent decrease in glutamine synthetase expression in the hippocampal astroglia of the triple transgenic Alzheimer's disease mouse model: mechanism for deficient glutamatergic transmission?Mol Neurodegen, 6 (2011), p. 55
Sydow A, Hochgräfe K, Könen S, et al (2016) Age-dependent neuroinflammation and cognitive decline in a novel Ala152Thr-Tau transgenic mouse model of PSP and AD. Acta Neuropathol Commun 4:17. doi: 10.1186/s40478-016-O281-z

1. A method for the treatment and/or prevention of Alzheimer's disease (AD) or a non-AD tauopathy, comprising administering a monoclonal antibody, or an antigen binding fragment thereof, that binds to an antigen comprising the sequence QGGYTMHQDQ (SEQ ID No. 1) and possesses at least one biological activity selected from inhibition of pathological hyperphosphorylation of Tau, reduction of the most neurotoxic amyloid-precursor protein (APP)-derived Aβ species (monomer and low-molecular weight oligomers), increase in task-induced Arc expression when compared to a proper control, significant neuroprotection in at least one of two different hippocampal-based behavioural tasks (Novel object recognition (NOR) and Object Place Recognition (OPR)), prevention of the loss in dendritic spine density, reduction of neuro inflammation, and normalization of LTP changes to a patient in need thereof.

2. The method of claim 1, wherein the monoclonal antibody, or an antigen binding fragment thereof does not change full-length tau levels when compared to a proper control.

3. The method of claim 1, wherein the monoclonal antibody, or an antigen binding fragment thereof binds to an antigen consisting of the sequence QGGYTMHQDQ (SEQ ID No. 1).

4. The method of claim 1, wherein said antibody or antigen binding fragment thereof comprises at least one human constant region.

5. The method of claim 4, wherein said constant region is the human IgGI/lgKappa constant region.

6. The method of claim 1, wherein said antibody or antigen binding fragment thereof is a humanized or resurfaced antibody.

7. The method of claim 1, wherein said antibody or antigen binding fragment thereof is a Fab, Fab′, F(ab′)2 or Fv fragment.

8. The method of claim 1, wherein said antibody is a bispecific antibody.

9. The method of claim 1, wherein the antibody or antigen binding fragment thereof is administered as a conjugate.

10. The method of claim 1 monoclonal antibody, or an antigen binding fragment thereof or the conjugate for use according to claims 1-9, wherein the AD is a genetic or sporadic form.

11. The method of claim 1, wherein the patient is administered a pharmaceutical composition comprising the monoclonal antibody, or an antigen binding fragment thereof or a conjugate comprising the monoclonal antibody, or an antigen binding fragment thereof and proper excipients.

12. The method of claim 11, wherein the pharmaceutical composition, further comprises a therapeutic agent.

13. The method of claim 12 wherein the therapeutic agent is selected from the group consisting of: Tau Aggregation/oligomerization Inhibitors; Kinase Inhibitors and Phosphatase Activators; Microtubule Stabilizers; activators of autophagy and proteasome-mediated clearance; reactive oxygen species (ROS) inhibitors, mitochondrial function enhancers; active and passive vaccination.
